(19) United States
(12) Patent Application Publication
(10) Pub. No.: US 2025/0213657 A1
(43) Pub. Date: Jul. 3, 2025

(54) Compositions and Methods for Treating Neurodegenerative Diseases and Disorders

(71) Applicant: Olfactive Ai, Inc., Mountain View, CA (US)

(72) Inventor: Christopher Hanson, Mountain View, CA (US)

(21) Appl. No.: 19/070,846

(22) Filed: Mar. 5, 2025

Related U.S. Application Data
(63) Continuation-in-part of application No. 18/980,129, filed on Dec. 13, 2024, now Pat. No. 12,290,550.
Application No. 18/980,129 is a continuation-in-part of application No. 18/892,760, filed on Sep. 23, 2024.
Application No. 18/892,760 is a division of application No. 18/615,150, filed on Mar. 25, 2024, now Pat. No. 12,102,664.
Application No. 18/615,150 is a continuation-in-part of application No. 18/430,796, filed on Feb. 2, 2024, now Pat. No. 12,115,134.
(63) Continuation-in-part of application No. 18/958,791, filed on Nov. 25, 2024.
Application No. 18/958,791 is a continuation of application No. 18/811,171, filed on Aug. 21, 2024, now Pat. No. 12,186,299.
Application No. 18/811,171 is a division of application No. 18/615,452, filed on Mar. 25, 2024, now Pat. No. 12,102,611.
(60) Provisional application No. 63/615,100, filed on Dec. 27, 2023.

(51) Int. Cl. A61K 38/26; A61K 31/085; A61K 31/202

(52) U.S. Cl. CPC A61K 38/26; A61K 31/085; A61K 31/202

(57) ABSTRACT

Disclosed herein are compounds and ligands, and compositions formed therewith, that modulate insulin and, some instances, dopamine secretion by activating endogenous receptors. Also disclosed herein are methods for using the compositions to treat neurodegenerative disorders, such as Parkinson's disease.

### CROSS-REFERENCES TO RELATED APPLICATIONS

This application is a continuation-in-part of U.S. application Ser. No. 18/980,129, filed on Dec. 13, 2024, which is a continuation-in-part of U.S. application Ser. No. 18/892,760, filed on Sep. 23, 2024, which is a divisional of U.S. application Ser. No. 18/615,150, filed on Mar. 25, 2024, now U.S. Pat. No. 12,102,664, which is a continuation-in-part of U.S. application Ser. No. 18/430,796, now U.S. Pat. No. 12,115,134, filed on Feb. 2, 2024, which claims the benefit of U.S. Provisional Application No. 63/615,100, filed on Dec. 27, 2023.

This application is also a continuation-in-part of U.S. application Ser. No. 18/958,791, filed on Nov. 25, 2024, which is a continuation application of U.S. application Ser. No. 18/811,171, filed on Aug. 21, 2024, now U.S. Pat. No. 12,186,299, which is a divisional application of U.S. application Ser. No. 18/615,452, filed on Mar. 25, 2024, now U.S. Pat. No. 12,102,611.

### FIELD OF THE INVENTION

The present invention relates to compositions and methods for treating physiological disorders. More particularly, the present invention relates to compositions and methods for treating neurodegenerative disease and disorders by modulating receptor activity.

### BACKGROUND OF THE INVENTION

As is well established, insulin is a potent anabolic hormone that exerts a variety of effects on many types of cells. Some of the main metabolic actions of insulin are stimulating glucose uptake in skeletal muscles and adipocytes, promoting glycogen synthesis in skeletal muscles, suppressing hepatic glucose production, and inhibiting lipolysis in adipocytes.

In most instances, insulin secretion is induced by pancreatic β-cells when glucagon-like peptide-1 (GLP-1) binds to and activates GLP-1 receptor proteins on endogenous gastrointestinal (GI) cells, such as enteroendocrine L-cells.

As is also well established, in metabolically impaired patients, abnormal insulin secretion and loss of cellular sensitivity to insulin signaling (insulin resistance) can occur, which principally affects liver, muscle, and adipose cells and is selective for glucose and lipid metabolism. Insulin resistance results in a reduction in insulin-mediated glucose uptake by endogenous cells and further results in compensatory hypersecretion of insulin by pancreatic β-cells to maintain homeostasis. The hypersecretion of insulin by β-cells typically leads to β-cell exhaustion and dysfunction, and ultimately impairment of insulin production by the β-cells (i.e., abnormal insulin secretion).

As is additionally well established, abnormal insulin secretion and associated insulin resistance are associated with various insulin-resistance-induced physiological disorders; particularly, type 2 diabetes mellitus. The prevalence of type 2 diabetes mellitus continues to rise worldwide as lifestyles associated with low energy expenditure and high caloric intake and, hence, metabolic dysfunction, are increasingly adopted, particularly in lower-income and developing countries. Indeed, it is estimated that the number of cases of type 2 diabetes mellitus is projected to rise from 830 million worldwide to 1.3 billion by 2050.

Recent studies also reflect that insulin resistance and, hence, hyperglycemia associated therewith, is also associated with Parkinson's disease development and progression through mitochondrial dysfunction, reactive oxygen species (ROS) overproduction, and increased alpha-synuclein (α-syn) production.

Recent studies thus also reflect that there is a strong correlation between abnormal insulin secretion and insulin resistance-associated disorders; particularly, type 2 diabetes mellitus, and Parkinson's disease.

Various entities have thus developed pharmaceutically active agents and compositions that are adapted to treat abnormal insulin secretion and associated insulin resistance. In view of the strong correlation between activation of the GLP-1 receptor proteins and insulin secretion, the pharmaceutically active agents and compositions are specifically adapted to activate the GLP-1 receptor proteins on endogenous gastrointestinal (GI) cells.

Such pharmaceutically active agents include semaglutide (Ozempic®, Rybelsus®, Wegovy®), dulaglutide (Trulicity®), exenatide (Bydureon BCise®, Byetta®), and liraglutide (Victoza®, Saxenda®).

The noted pharmaceutically active agents (referred to hereinafter as “GLP-1 analogs”) mimic endogenous GLP-1 and are adapted to activate the GLP-1 receptors on endogenous GI cells and, hence, function as GLP-1 receptor agonists.

Although the GLP-1 analogs can effectively activate GLP-1 receptors on pancreatic β-cells and, hence, can induce insulin secretion and, thereby, effectively treat various physiological disorders, there are several drawbacks and disadvantages associated with administration of the GLP-1 analogs to patients.

A major drawback associated with the administration of the GLP-1 analogs to patients is the high risk of adverse pathological events. One such adverse pathological event is hypoglycemia (i.e., low blood glucose), which can, and often will, present in patients that are also taking or being administered commonly prescribed antidiabetic agents, such as basal insulin and sulfonylureas.

There is also a high risk of induced production of anti-GLP-1 antibodies and binding of endogenous GLP-1 and the GLP-1 analogs to the anti-GLP-1 antibodies, which can, and often will, induce adverse immune responses.

A further major drawback associated with the administration of GLP-1 analogs to individuals are the significant side effects that are often presented by the individuals, including nausea, vomiting, diarrhea, abdominal pain, and constipation.

Since most GLP-1 analogs are administered to patients via a subcutaneous injection, a further drawback associated with GLP-1 analog administration is the pain and discomfort associated with the often-prescribed weekly injections.

Although the GLP-1 analogs developed by Novo Nordisk, which are marketed under the tradename Rybelsus, can also be delivered orally, a significantly greater dose of the Rybelsus GLP-1 analog must be orally administered to an individual to match the pharmacokinetics of the Novo Nordisk injectable GLP-1 analog, which is marketed under the tradename Ozempic, i.e., individuals must be orally administered approximately 100.0 mg/week of the Rybelsus GLP-1 analog to match the efficacy of the typically prescribed 0.5 mg/week of the injectable Ozempic GLP-1 analog.

A further major drawback associated with the administration of GLP-1 analogs to individuals is the cost. Indeed, the costs, at present, for a thirty (30) day supply of Ozempic and Rybelsus are approximately $1000.00 and $1200.00, respectively.

As is also well established, insulin secretion can also be induced by pancreatic β-cells when gastric inhibitory polypeptide (GIP) binds to and activates GIP receptor proteins on the pancreatic β-cells.

Although GIP also induces insulin secretion, there are similarly several drawbacks and disadvantages associated with solely activating the GIP receptor proteins on the pancreatic β-cells.

A major disadvantage associated with solely activating the GIP receptor proteins on the pancreatic β-cells is that GIP also induces glucagon secretion from pancreatic β-cells. Since glucagon is a hyperglycemic compound that increases blood sugar when secreted, the increase in glucagon secretion induced by GIP limits its therapeutic potential for treating abnormal insulin secretion.

To address the above noted disadvantage associated with solely activating the GIP receptor proteins on the pancreatic β-cells, Eli Lilly has recently developed a dual GLP-1/GIP analog that activates GLP-1 and GIP receptors on pancreatic β-cells.

The dual GLP-1/GIP analog, i.e., tirzepatide, which is marketed under the tradenames Mounjaro® and ZepBound®, provides the beneficial metabolic activity induced by both GLP-1 and GIP in a synergistic manner without a clinically significant increase in glucagon secretion.

Many of the drawbacks and disadvantages associated with administration of the GLP-1analogs to patients are, however, also associated with administration of the dual GLP-1/GIP analog to patients.

Such drawbacks and disadvantages include the significant side effects that are often presented by the patients, including nausea, vomiting, diarrhea, abdominal pain, kidney problems and constipation, and high cost.

There is thus a need for compounds and ligands; particularly, natural compounds and ligands, and compositions comprising same, for treating physiological disorders; particularly, insulin-resistance-induced physiological disorders, such as type 2 diabetes mellitus, and neurodegenerative disorders, such as Parkinson's disease, which substantially reduce or overcome the drawbacks and disadvantages associated with administration (or delivery) of GLP-1 analogs and dual GLP-1/GIP analogs to a patient presenting with a physiological disorder.

It is thus one object of the present invention to provide natural compounds and ligands, and compositions comprising same, for treating physiological disorders; particularly, insulin-resistance-induced physiological disorders, such as type 2 diabetes mellitus, and neurodegenerative disorders, such as Parkinson's disease, which substantially reduce or overcome the drawbacks and disadvantages associated with administration of GLP-1 analogs and dual GLP-1/GIP analogs to a patient presenting with a physiological disorder.

It is another object of the present invention to provide natural compounds and ligands, and compositions comprising same, which effectuate OR-mediated, free fatty acid receptor-mediated, and transient potential ion channel-mediated secretion of endogenous GLP-1 PYY and GIP in a patient presenting with a physiological disorder, without the undesirable side effects associated with delivery of a GLP-1 analog and/or a dual GLP-1/GIP analog to patients.

It is another object of the present invention to provide natural compounds and ligands, and compositions comprising same, which, when administered to a patient, effectively and safely modulate the patient's systemic insulin secretion in vivo, and can be administered to the patient via oral, sublingual, inhalation, intranasal, epidural, intracerebral, transdermal, topical, and injection administration means.

It is another object of the present invention to provide natural compounds and ligands, and compositions comprising same, which, when administered to a patient effectively and safely modulate dopamine synthesis and, thereby, dopamine secretion in vivo, and can be administered to the patient via oral, sublingual, inhalation, intranasal, epidural, intracerebral, transdermal, topical, and injection administration means.

It is another object of the present invention to provide natural compounds and ligands, and compositions comprising same, which, when administered to a patient presenting with an insulin-resistance-induced physiological disorder; particularly, type 2 diabetes mellitus, effectively and safely modulate the patient's insulin secretion in vivo, whereby the insulin-resistance-induced physiological disorder is effectively treated and at least one risk factor associated with the insulin-resistance-induced physiological disorder is ameliorated with minimal, if any, adverse side effects.

It is another object of the present invention to provide natural compounds and ligands, and compositions comprising same, which, when administered to a patient presenting with a neurodegenerative disorder; particularly, Parkinson's disease, effectively and safely modulate the patient's insulin secretion in vivo, whereby the neurodegenerative disorder is effectively treated and at least one risk factor associated with the neurodegenerative disorder is ameliorated with minimal, if any, adverse side effects.

It is another object of the present invention to provide natural compounds and ligands, and compositions comprising same, which, when administered to a patient presenting with a neurodegenerative disorder; particularly, Parkinson's disease, effectively and safely modulate the patient's dopamine secretion in vivo, whereby the neurodegenerative disorder is effectively treated and at least one risk factor associated with the neurodegenerative disorder is ameliorated with minimal, if any, adverse side effects.

It is another object of the present invention to provide natural compounds and ligands, and compositions comprising same, which, when administered to a patient presenting with an insulin-resistance-induced physiological disorder; particularly, type 2 diabetes mellitus, and a neurodegenerative disorder; particularly, Parkinson's disease, effectively and safely modulate the patient's insulin and dopamine secretion in vivo, whereby the insulin-resistance-induced physiological disorder and/or neurodegenerative disorder is effectively treated and at least one risk factor associated with the insulin-resistance-induced physiological disorder or neurodegenerative disorder is ameliorated with minimal, if any, adverse side effects.

### SUMMARY OF THE INVENTION

The present invention is directed to compositions and methods for treating physiological disorders; particularly, neurodegenerative diseases and disorders, and ameliorating physiological risk factors and seminal pathophysiological effects associated therewith.

In some embodiments of the invention, there are thus provided compositions for treating a neurodegenerative disorder presented by a patient.

In some embodiments, a composition for treating a neurodegenerative disorder presented by a patient comprises:
(i) at least a first receptor activating compound adapted to bind to and activate at least a first receptor selected from the group comprising olfactory receptor family 51 subfamily E member 1 (OR51E1) and olfactory receptor family 2 subfamily C member 1 (OR2C1), and
(ii) at least a second receptor activating compound adapted to bind to and activate at least a second receptor selected from the group comprising free fatty acid receptor 1 (FFAR1) and free fatty acid receptor 4 (FFAR4),
the composition adapted to modulate insulin secretion in vivo, whereby the neurodegenerative disorder is treated when the composition is delivered to the patient.

In a preferred embodiment, the composition is further adapted to ameliorate at least one risk factor associated with the neurodegenerative disorder when the composition is delivered to the patient.

In some embodiments, the composition is further adapted to ameliorate at least one seminal pathophysiological effect associated with the neurodegenerative disorder when the composition is delivered to the patient.

In a preferred embodiment, the neurodegenerative disorder comprises Parkinson's disease.

In a preferred embodiment, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the composition.

In some embodiments, the eugenol comprises an EC₅₀ value in the composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the composition.

In some embodiments, the 3-methylpentanoic acid comprises an EC₅₀ value in the composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the composition.

In some embodiments, the lauric acid comprises an EC₅₀ value in the composition in the range of approximately 0.05 μM to approximately 50.0 μM.

In some embodiments, the composition further comprises at least a third receptor activating compound adapted to bind to and activate at least a third receptor selected from the group comprising olfactory receptor family 2 subfamily B member 11 (OR2B11), olfactory receptor family 2 subfamily J member 3 (OR2J3) and transient receptor potential cation channel subfamily A member 1 (TRPA1).

In some embodiments, the third receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the composition.

In some embodiments, the cinnamaldehyde comprises an EC₅₀ value in the composition in the range of approximately 0.1 μM to approximately 2500.0 μM.

In a preferred embodiment, the first receptor activating compound is adapted to induce at least 50% activation of at least olfactory receptor olfactory receptor OR51E1 in vivo when the composition is delivered to the patient.

In some embodiments of the invention, a composition for treating a neurodegenerative disorder presented by a patient comprises:
(i) at least a first receptor activating compound adapted to bind to and activate at least a first receptor selected from the group comprising olfactory receptor family 51 subfamily E member 1 (OR51E1), olfactory receptor family 1 subfamily A member 1 (OR1A1), olfactory receptor family 2 subfamily C member 1 (OR2C1) and olfactory receptor family 10 subfamily J member 5 (OR10J5),
(ii) at least a second receptor activating compound adapted to bind to and activate at least a second receptor selected from the group comprising free fatty acid receptor 1 (FFAR1), free fatty acid receptor 4 (FFAR4), olfactory receptor family 2 subfamily W member 1 (OR2W1), olfactory receptor family 2 subfamily B member 11 (OR2B11), olfactory receptor family 2 subfamily J member 3 (OR2J3) and transient receptor potential cation channel subfamily A member 1 (TRPA1), and
(iii) at least a third receptor activating compound adapted to bind to and activate short transient receptor potential channel 4 (TRPC4),
the composition adapted to modulate insulin and dopamine secretion in vivo, whereby the neurodegenerative disorder is treated when the composition is delivered to a patient.

In a preferred embodiment, the composition is further adapted to ameliorate at least one risk factor associated with the neurodegenerative disorder when the composition is delivered to the patient.

In some embodiments, the composition is further adapted to ameliorate at least one seminal pathophysiological effect associated with the neurodegenerative disorder when the composition is delivered to the patient.

In a preferred embodiment, the neurodegenerative disorder similarly comprises Parkinson's disease.

In some embodiments, the first receptor activating compound comprises a first compound selected from the group comprising eugenol, 3-methylpentanoic acid and geraniol.

In some embodiments, the first compound comprises eugenol.

In some embodiments, the eugenol comprises an EC₅₀ value of at least approximately 0.1 μM in the composition.

In some embodiments, the eugenol comprises an EC₅₀ value in the composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the composition.

In some embodiments, the 3-methylpentanoic acid comprises an EC₅₀ value in the composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the composition.

In some embodiments, the lauric acid comprises an EC₅₀ value in the composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the composition.

In some embodiments, the cinnamaldehyde comprises an EC₅₀ value in the composition in the range of approximately 0.1 μM to approximately 2500.0 μM.

In some embodiments, the third receptor activating compound comprises a fourth compound selected from the group comprising (−)-englerin A and choline.

In some embodiments, the fourth compound comprises (−)-englerin A.

In some embodiments, the (−)-englerin A comprises an EC₅₀ value of at least approximately 0.1 μM in the composition.

In some embodiments, the (−)-englerin A comprises an EC₅₀ value in the composition in the range of approximately 0.003 μM to approximately 0.1 μM.

In a preferred embodiment, the first receptor activating compound is adapted to induce at least 50% activation of at least olfactory receptor olfactory receptor OR51E1 in vivo when the composition is delivered to the patient.

In a preferred embodiment, the third receptor activating compound is adapted to induce at least 50% activation of transient receptor TRPC4 in vivo when the GLP-1/GIP secretion composition is delivered to the patient.

In some embodiments of the invention, there are also provided compositions for treating a neurodegenerative disorder and an insulin-resistance-induced physiological disorder presented by a patient.

In some embodiments of the invention, a composition for treating a neurodegenerative disorder and an insulin-resistance-induced physiological disorder presented by a patient comprises:
(i) at least a first receptor activating compound adapted to bind to and activate at least a first receptor selected from the group comprising olfactory receptor family 51 subfamily E member 1 (OR51E1), olfactory receptor family 1 subfamily A member 1 (OR1A1), olfactory receptor family 2 subfamily C member 1 (OR2C1) and olfactory receptor family 10 subfamily J member 5 (OR10J5), and
(ii) at least a second receptor activating compound adapted to bind to and activate at least a second receptor selected from the group comprising free fatty acid receptor 1 (FFAR1), free fatty acid receptor 4 (FFAR4), olfactory receptor family 2 subfamily W member 1 (OR2W1), olfactory receptor family 2 subfamily B member 11 (OR2B11), olfactory receptor family 2 subfamily J member 3 (OR2J3) and transient receptor potential cation channel subfamily A member 1 (TRPA1),
the composition adapted to induce GLP-1 and GIP secretion, and, thereby, increased insulin secretion in vivo, whereby the neurodegenerative disorder and insulin-resistance-induced physiological disorder are treated when the composition is delivered to a patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the composition.

In some embodiments, the eugenol comprises an EC₅₀ value in the composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the composition.

In some embodiments, the 3-methylpentanoic acid comprises an EC₅₀ value in the composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the composition.

In some embodiments, the lauric acid comprises an EC₅₀ value in the composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the composition.

In some embodiments, the cinnamaldehyde comprises an EC₅₀ value in the composition in the range of approximately 0.1 μM to approximately 2500.0 μM.

In a preferred embodiment, at least one of the first receptor activating compounds is adapted to induce at least 50% activation of at least olfactory receptor OR51E1 in vivo when the composition is delivered to the patient.

In some embodiments of the invention, there are thus also provided methods for treating a neurodegenerative disorder presented by a patient.

In some embodiments, a method for treating a neurodegenerative disorder presented by a patient comprises the steps of:
(a) providing one of the compositions referenced above; and
(b) delivering the composition to the patient, wherein insulin secretion of the patient is modulated in vivo, whereby the neurodegenerative disorder is effectively treated.

In some embodiments, a method for treating a neurodegenerative disorder presented by a patient comprises the steps of:
(a) providing one of the compositions referenced above; and
(b) delivering the composition to the patient, wherein insulin and dopamine secretion of the patient are modulated in vivo, whereby the neurodegenerative disorder is effectively treated.

In some embodiments of the invention, there are also provided methods for treating a neurodegenerative disorder and an insulin-resistance-induced physiological disorder presented by a patient.

In some embodiments, a method for treating a neurodegenerative disorder and an insulin-resistance-induced physiological disorder presented by a patient comprises the steps of:
(a) providing the dual disorder treatment composition referenced above; and
(b) delivering the composition to the patient, wherein insulin and dopamine secretion of the patient are modulated in vivo, whereby the neurodegenerative disorder and insulin-resistance-induced physiological disorder are effectively treated.

In a preferred embodiment, the neurodegenerative disorder treated by the noted compositions comprises Parkinson's disease.

In some embodiments of the invention, the insulin-resistance-induced physiological disorder treated by the dual disorder treatment composition comprises type 2 diabetes mellitus.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a schematic illustration of receptor-mediated activation of GLP-1 secretion from endogenous gastrointestinal cells; and

FIG. 2 is a schematic illustration of receptor-mediated activation of GIP secretion from endogenous gastrointestinal cells.

### DETAILED DESCRIPTION OF THE PREFERRED EMBODIMENT

Before describing the present invention in detail, it is to be understood that this invention is not limited to particularly exemplified compounds, compositions or methods, as such may, of course, vary. Thus, although a number of compounds, compositions and methods similar or equivalent to those described herein can be used in the practice of the present invention, the preferred compounds, compositions and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a”, “an” and “the” include plural referents unless the content clearly dictates otherwise. Definitions

The term “olfactory receptor (OR)” as used herein, means and includes an olfactory receptor that is a seminal component of the chemosensory organs responsible for olfaction. The term “olfactory receptor” as used herein, also means, and includes, trace amine associated receptors, vomeronasal receptors, formyl peptide receptors, membrane guanylyl cyclase, subtype GC-D receptors; and G-protein coupled receptors, such as G-protein coupled taste receptors. Olfactory receptors can also include hybrid receptors synthesized from the above-noted olfactory receptors.

The term “ectopic olfactory receptor”, as used herein, means and includes an olfactory receptor that is present in organs, tissue, and/or cells that is a seminal component of physiological processes outside of olfaction and, in some instances, indirectly involved with olfactory-mediated processes.

The terms “olfaction” and “olfactory reception” are used interchangeably herein, mean and include the interaction of a composition (or formulation) with an olfactory receptor coupled to a cell signaling pathway. The composition can also be defined as an “odorant” and may be airborne (i.e., volatile) and/or in solution.

The term “free fatty acid receptor”, as used herein, means and includes a transmembrane cell surface receptor that is adapted and configured to bind to fatty acids and induce cell signaling processes in response to the binding of the fatty acids.

The term “transient receptor potential ion channel”, as used herein, means and includes a transmembrane ion channel that is adapted and configured to modulate ion entry into an endogenous cell, such as Ca²⁺entry, and, thereby, induce cell signaling process when a compound or ligand binds to the ion channel.

The term “neurotransmitter,” as used herein, means and includes any molecular compound that is produced, secreted and regulated by endogenous cells that facilitates communication by and between cells, whereby physiological activities are induced. The term “neurotransmitter” thus means and includes, without limitation, serotonin, dopamine, glutamate, gamma-aminobutyric acid (GABA), glycine, norepinephrine, histamine, substance P and acetylcholine (ACh).

The term “neurotransmission,” as used herein, means and includes the plurality of signaling processes between neurons via neurotransmitters, including, without limitation, neurotransmitter production and synthesis, modulation of neuron action potentials, modulation of neuronal membrane depolarization, synaptic vesicle docking, neurotransmitter secretion, extracellular neurotransmitter diffusion, neurotransmitter reuptake, ion channel activation and receptor activation.

The term “dysregulation,” as used herein in connection with a neurotransmitter, means and includes abnormality or impairment in the synthesis, production and/or neurotransmission of a neurotransmitter and, hence, abnormality or impairment in biological processes modulated by the neurotransmitter.

The term “modulation,” as used herein in connection with a neurotransmitter, means and includes, without limitation, activating and/or regulating a physiological activity, preferably, multiple physiological activities associated with the neurotransmitter, including, without limitation, regulation of (i) production and, hence, release of the neurotransmitter via activation of a receptor, e.g., olfactory receptor family 51 subfamily E member 1 (OR51E1), and/or transient receptor, e.g., transient receptor potential cation channel subfamily A member 1 (TRPA1), (ii) binding of the released neurotransmitter to one or more receptors, such a D1 receptor for dopamine, (iii) synthesis of the neurotransmitter, (iv) alteration(s) of intracellular signaling pathways associated with the neurotransmitter and (v) alteration(s) of ligand-gated ion channel activity associated with the neurotransmitter.

The term “insulin resistance”, as used herein, means, and includes a condition in which insulin exerts a biological effect that is lower than expected, due to defects in insulin-stimulated glucose uptake; particularly, in glycogen synthesis and, to a lesser extent, glucose oxidation.

The term “insulin-resistance-induced physiological disorder”, as used herein, thus means and includes a physiological disease and/or a physiological disorder characterized by metabolic dysfunction and conditions associated therewith including, but not limited to, dysfunction of glucose metabolism and attendant insulin resistance.

The term “modulation,” as used herein in connection with insulin, means and includes, without limitation, activating and/or regulating at least one cellular process relating to production, synthesis and transmission of insulin, including, but not limited to (i) production and, hence, release of the insulin via activation of a receptor, e.g., GLP-1 receptor and GIP receptor activation, (ii) binding of the released insulin to insulin receptors, (iii) synthesis of the insulin, (iv) alteration(s) of intracellular signaling pathways associated with insulin synthesis and secretion and (v) alteration(s) of cellular sensitivity to extracellular insulin.

The term “modulation,” as used herein in connection with dopamine, thus, also means and includes, without limitation, activating and/or regulating at least one cellular process relating to production, synthesis and neurotransmission of dopamine, including, but not limited to (i) activating and/or regulating at least one receptor and/or transient receptor including, but not limited to, olfactory receptor family 2 subfamily A member 4 (OR2A4), olfactory receptor family 51 subfamily E member 1 (OR51E1), olfactory receptor family 51 subfamily E member 2 (OR51E2), transient receptor potential channel 4 (TRPC4), taste 1 receptor member 3 (TAS1R3) and trace amine-associated receptor 5 (TAAR5) of a cell population, whereby ion-induced depolarization of the cell population is initiated and induces release of dopamine from the cell population via vesicular exocytosis, (ii) and regulating binding of the released dopamine to at least one dopamine receptor including, but not limited to, D₁, D₂, D₃, D₄ and D₅ receptors, and at least one ligand-gated ion channel including, but not limited to, GABAA-gated channels, AMPA channels and NMDA channels.

The term “modulation,” as used herein in connection with a neurotransmitter thus includes, without limitation, neurotransmitter production, restricted neurotransmitter production and presynaptic neuronal uptake of the neurotransmitter.

The term “agonist” as used herein, means, and includes any molecule which binds to a receptor on a cell, wherein the binding to the receptor can potentially lead to subsequent changes in the cell's functions. When an agonist binds to a sufficient number of receptors, the receptors can activate seminal processes in the cell.

The term “antagonist”, as used herein, means and includes a molecule, which binds to a receptor on a cell and inhibits the receptor from activating processes in the cell. The inhibition of the receptor can include competitive binding against agonists (when an antagonist is bound, agonists cannot bind to the receptor) and allosteric effects (when the antagonist binds, agonists can still bind the receptor, but cannot activate the receptor).

The term “endocrine factor” as used herein, means and includes any molecular compound that is produced and secreted by endogenous cells and induces biological activity at a biological tissue site. The term “endocrine factor” thus means and includes, without limitation, glucagon-like peptide-1 (GLP-1), gastric inhibitory polypeptide (GIP), peptide Y-Y (PYY), ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, enteroglucagon, galanin, leptin, motilin, amylin, neuropeptide Y (NPY), pancreatic polypeptide, substance P, oxyntomodulin, and somatostatin.

The term “compound”, as used herein, means and includes any composition of matter comprising two or more chemical elements. According to the invention, in some instances, the terms “compound” and “ligand” are synonymous and used interchangeably herein.

The term “compound” thus means and includes, without limitation, the following natural compounds and ligands (referred to herein as “receptor activating compounds and ligands): pentanoic acid, pentanol, 3-methylpentanoic acid, 4-methylpentanoic acid, 3-methyl-2,4 nonanedione, eugenol, farnesol, farnesyl thiosalicylic acid, acrolein, formalin, hydrogen peroxide, coumarin, dicyclohexyl disulfide, nonanoic acid, octanoic acid, 2-nonanoic acid, butyric acid, 2-heptanone, heptanoic acid, decanoic acid, tetradecanoic acid, trans-2-decenoic acid, tridecanoic acid, undecanoic acid, methyl eugenol, methyl salicylate, (+)-menthol, eugenyl acetate, 2,4-dinitrotoluene, 4-hydroxynonenal, hexanoic acid, 2-ethylhexanoic acid, 2-ethyl-3,5-dimethylpyrazine, pyrazine, dimethyl disulfide, methyl furfuryl disulfide, propanal, butyl butyryl lactate, isovaleric acid, propionic acid, methanoic acid, coumarin, acrolein, helional, lilial, β-ionone, androstenone, androstadienone, caramel furanone, 3-phenyl propyl propionate, ethyl vanillin, 2-ethyl-fencol, N-amyl acetate, eugenol acetate, sandalwood, S-(−)-citronellol, (−)-citronellol, hydroxycitronellal, citral, S-(−)-citronellal, geraniol, estragole, neroli, heptanol, octanol, nonanal, (+)-carvine, (−)-carvone, (+)-carvone, choline, (−)-englerin A, cyclohexyl salicylate (CHS), sandacanol, brazzein, monellin, xylitol, sorbitol, trimethylamine (TMA), N,N-dimethylethylamine, linalool, bourgeonal, acetophenone, amyl butyrate, nonanethiol, allyl phenyl acetate, N-amyl acetate, muscone, isoeugenol, eugenol methyl ether, heptanol, hexanol, hexanal, hexyl acetate, 1-hexanol, 1-heptanol, 2-heptanone, octanal, octanol, 1-octanol, 1-octanal, musk ketone, (+)-dihydrocarvone, α-cedrene, celery ketone, anis aldehyde, vanillin, guaiacol, thujopsene, hydroxymethylpentylcyclohexenecarboxaldehyde (lyral), allyl phenylacetate, allyl isothiocyanate, benzyl acetate, 3,4-hexanedione, cis-3-hexen-1-ol, quinoline, ethyl heptanoate, methyl octanoate, nonanal, 1-nonanol, 2-nonanol, 3-octanone, 3-nonanone, decyl aldehyde, (E)-non-2-enal, 2-ethyl-3,5-dimethylpyrazine, 3-methylbut-2-ene-1-thiol, (2E,6Z)-nona-2,6-dienalcitral, ethyl octanoate, octanoate, p-mentha-8-thiol-3-one, β-myrcene, γ-decalactone, (S)-(+)-carvone, dihydrojasmone, dicyclohexyl disulfide, cinnamaldehyde, spearmint, coffee difuran, quinoline, butyl anthranilate 2,2-dithiodimethylenedifuran, ethyl hexanoate, limonene, α-terpineol, eugenol (3E,5Z)-undeca-1,3,5-triene, acetate, butyrate, nicotinic acid, long-chain free fatty acids (e.g., palmitic acid and stearic acid), medium-chain free fatty acids (e.g., caproic acid (C6:0), caprylic acid (C8:0), capric acid (C10:0), and lauric acid (C12:0)), and omega-3 polyunsaturated fatty acids (e.g., alpha-linoleic acid, docosahexaenoic acid and eicosatetraenoic acid).

The term “compound” also means and includes any composition of matter included in the Food and Drug Administration's (FDA's) generally recognized as safe (GRAS) database.

The term “natural,” as used herein in connection with a compound or ligand, and a composition of the invention formed therewith, means and includes a compound or ligand that exists or is synthesized in nature without intervention, including, by way of example, a food molecule.

The term “natural,” as used herein in connection with a compound or ligand, and a composition of the invention formed therewith, also means and includes a compound or ligand that originally existed or was synthesized in nature without intervention and is subjected to processing without altering the chemical structure of the compound, such as chemical purification and isolation processes and the formulation of compositions from two or more compounds.

The terms “composition”, “formulation”, “olfactory composition” and “olfactory formulation” are used interchangeably herein, mean and include any compound or combination of compounds that can interact with and modulate at least one olfactory receptor and/or ectopic olfactory receptor and/or free fatty acid receptor and/or transient receptor potential ion channel.

The terms “express” and “expression” as used interchangeably herein, mean, and include the production of a protein product from the genetic information contained within a nucleic acid sequence.

The term “upregulation”, as used herein, means, and includes the increased production of a protein product from the genetic information contained within a nucleic acid sequence.

The term “downregulation”, as used herein, means, and includes the decreased production of a protein product from the genetic information contained within a nucleic acid sequence.

The terms “prevent” and “preventing” are used interchangeably herein, and mean and include precluding a disease, physiological disorder, or pathological condition presented by a subject or patient. The term does not require an absolute preclusion of the disease or condition. Rather, this term includes decreasing the chance for disease occurrence and recurrence.

The terms “prevent” and “preventing” also mean and include reducing the frequency or severity of a disease, physiological disorder or pathological condition presented by a subject or patient.

The terms “treat,” “treatment” and “treating” are used interchangeably herein, and mean and include management of a disease, physiological disorder or pathological condition presented by a subject or patient to cure, ameliorate, stabilize, or prevent the disease, physiological disorder or pathological condition. The terms “treat” and “treating” include “active treatment”, i.e., treatment intended to cure or stabilize a disease, physiological disorder or pathological condition, and “causal treatment”, i.e., treatment intended to abate or prevent the cause of the associated disease, physiological disorder or pathological condition.

The terms “treat,” “treatment” and “treating” further include “palliative treatment”, i.e., treatment intended to relieve the symptoms a disease, physiological disorder or pathological condition, “preventative treatment”, i.e., treatment intended to minimize or partially or completely inhibit the development of the associated disease, physiological disorder or pathological condition, and “supportive treatment”, i.e., treatment employed to supplement another treatment modality directed toward curing, ameliorating, stabilizing, or preventing the associated disease, pathological condition, or disorder.

The terms “delivery” and “administration” are used interchangeably herein, and mean and include providing a composition (or formulation), through any method appropriate to deliver the composition (or formulation) to a subject. According to the invention, such administration means includes, without limitation, oral, sublingual, inhalation, intranasal, epidural, intracerebral, transdermal, topical, and injection administration means.

The term “IC₅₀”, as used herein, means, and includes the concentration of an agonist, antagonist, compound and/or ligand, which, after delivery, inhibits or attenuates at least 50% of a biological and/or physiological process.

In some embodiments, the term “IC₅₀” refers to the concentration of a modulator (e.g., an antagonist or compound), which, after delivery, abates, inhibits or attenuates at least 50% of a biological and/or physiological process, e.g., α-synuclein (α-syn) protein production, reactive oxygen species (ROS) overproduction, and neuroinflammatory processes, e.g., maladaptive expression of pro-inflammatory cytokines.

The term “EC₅₀”, as used herein, means, and includes the concentration of an agonist, compound and/or ligand, which, after delivery to a subject, induces at least 50% activation of a biological and/or physiological process.

In some embodiments, the term “adapted”, as used in connection with a “compound”, “ligand”, “agonist”, and “antagonist”, means the “compound”, “ligand”, “agonist” and/or “antagonist” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonergic receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κβ (NK-κβ) and an NADPH oxidase (NOX) by virtue of the concentration, i.e., IC₅₀ or EC₅₀ value, of the “compound”, “ligand”, “agonist”, or “antagonist.”

In some embodiments, the term “adapted”, as used in connection with a “composition” and “formulation” also means the “composition” and/or “formulation” is capable of inducing or attenuating one or more biological and/or physiological processes or activities, including, without limitation, (i) activating or antagonizing a receptor, including, without limitation, adipose olfactory receptors, central nervous system (CNS) olfactory receptors, cardiovascular olfactory receptors, trace amine-associated receptors, gastrointestinal (GI) olfactory receptors, free fatty acid receptors, transient receptor potential ion channels, dopaminergic neuron olfactory receptors and serotonergic receptors and/or (ii) inducing or attenuating synthesis, secretion and transmission of a molecule or macromolecule, including, without limitation, β-secretase (BACE1), glucagon-like peptide-1 (GLP-1), peptide Y-Y (PYY), gastric inhibitory polypeptide (GIP), serotonin (5-HT), dopamine, secretin, prostaglandin E₂, vasoactive intestinal protein (VIP), nuclear factor κβ (NK-κβ) and an NADPH oxidase (NOX) by virtue the concentration, i.e., IC₅₀ or EC₅₀ value, of a “compound” or “ligand” contained in the “composition” or “formulation.”

In some embodiments, the term “adapted”, as used in connection with a “composition” and “formulation” also means the “composition” and/or “formulation” is capable of inducing or abating one or more biological and/or physiological processes or activities referenced above by virtue of the concentrations, i.e., IC₅₀ or EC₅₀ values, of two (2) or more “compounds”, “ligands”, “agonists”, or “antagonists” in the “composition” or “formulation.”

The term “comprise” and variations of the term, such as “comprising” and “comprises”, means “including, but not limited to” and is not intended to exclude, for example, other compounds, ligands or method steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention.

The present invention is directed to compositions and methods for treating neurodegenerative disorders; particularly, Parkinson's disease, and insulin-resistance-induced physiological disorders; particularly, type 2 diabetes mellitus, by modulating receptor activity and, thereby, insulin secretion and, in some embodiments, dopamine secretion.

As discussed above, various entities have developed GLP-1 analogs that mimic endogenous GLP-1 alone, and dual GLP-1/GIP analogs that mimic both endogenous GLP-1 and GIP in combination, which, when delivered to a patient, modulate secretion of endogenous GLP-1 and GIP, and, thereby, insulin secretion.

As also discussed above, the GLP-1 analogs activate the GLP-1 receptor on pancreatic β-cells and the dual GLP-1/GIP analogs activate both GLP-1 receptor and GIP receptor on pancreatic β-cells to modulate insulin secretion by the pancreas.

Although the GLP-1 analogs and dual GLP-1/GIP analogs can effectively modulate insulin secretion, as also discussed in detail above, there are several drawbacks and disadvantages associated with administration of GLP-1 analogs and dual GLP-1/GIP analogs to patients, including, a high risk of hypoglycemia, adverse side effects, and high costs.

As discussed in detail below, Applicant has developed compositions, which, when delivered to a patient, effectively and safely modulate endogenous GLP-1 and GIP secretion and, thereby, insulin secretion in vivo, which (i) overcome the drawbacks and disadvantages associated with delivery of GLP-1 analogs, which merely mimic endogenous GLP-1, and dual GLP-1/GIP analogs, which merely mimic endogenous GLP-1 and GIP, and (ii) can be readily employed to treat a multitude of physiological disorders.

Although the compositions of the invention are described primarily in connection with the treatment of neurodegenerative diseases and disorders and underlying causes thereof, use of the compositions is not limited solely to the treatment of neurodegenerative diseases and disorders, and underlying causes thereof. As set forth in Applicant's U.S. application Ser. Nos. 18/430,796 and 18/892,760, which are expressly incorporated by reference herein in their entirety, the compositions can also be employed to effectively treat various insulin-resistance-induced physiological disorders, such as type 2 diabetes mellitus.

As will readily appreciated by one having ordinary skill in the art, the compositions can also be employed to effectively treat a multitude of other physiological diseases and disorders, including, without limitation, cardiovascular diseases and disorders, e.g., atherosclerosis, liver diseases and disorders, e.g., fatty liver disease, psychopathological diseases and disorders, reproductive diseases and disorders, and immune diseases and disorders.

As discussed in detail herein, in preferred embodiment, the compositions of the invention comprise at least one natural compound or ligand that is adapted to bind to and activate (and, hence, modulate) at least one receptor, e.g., an ectopic olfactory receptor and/or free fatty acid receptor and/or transient receptor potential ion channel, whereby GLP-1 and/or peptide Y-Y (PYY) and/or GIP secretion is induced and, thereby, insulin secretion is modulated in vivo.

As also discussed in detail herein, in some embodiments, the compositions of the invention also comprise at least one natural compound or ligand that is adapted to bind to and activate (and, hence, modulate) at least one receptor, e.g., a transient receptor, whereby dopamine synthesis is induced and, thereby, dopamine secretion is also modulated in vivo.

### Receptor Activating Compounds and Ligands

According to the invention, suitable natural compounds and ligands, which are adapted to modulate insulin secretion via induced receptor activity, include, without limitation, the natural receptor activating compounds and ligands referenced above.

As discussed in detail herein, in some embodiments of the invention, the preferred natural receptor activating compounds and ligands of the invention that are adapted to modulate insulin secretion via induced receptor activity, include, without limitation, 3-methylpentanoic acid, 4-methylpentanoic acid, 3-methyl-2,4 nonanedione, eugenol, farnesol, β-ionone, isovaleric acid, propionic acid, acrolein, formalin, coumarin, nonanoic acid, octanoic acid, 2-heptanone, 4-hydroxynonenal, butyl butyryl lactate, isovaleric acid, acetate, butyrate, nicotinic acid, S-(−)-citronellol, (−)-citronellol, citral, S-(−)-citronellal, geraniol, estragole, neroli, heptanol, octanol, nonanal, hexanal, hexyl acetate, 1-hexanol, 1-heptanol, 1-octanal, musk ketone, (+)-dihydrocarvone, α-cedrene, thujopsene, lyral, allyl phenylacetate, allyl isothiocyanate, benzyl acetate, 3,4-hexanedione, cis-3-hexen-1-ol, 3-octanone, 2-ethyl-3,5-dimethylpyrazine, dicyclohexly disulfide, cinnamaldehyde, spearmint, coffee difuran, palmitic acid, stearic acid, caproic acid, caprylic acid, capric acid, lauric acid, alpha-linoleic acid, docosahexaenoic acid and eicosatetraenoic acid.

As also discussed in detail herein, several of the natural compounds and ligands referenced above; specifically, eugenol, farnesol, 3-methylpentanoic acid, 4-methylpentanoic acid, butyl butyryl lactate, nonanoic acid and isovaleric acid, are also adapted to modulate dopamine secretion via induced receptor activity.

As additionally discussed in detail herein, in some embodiments of the invention, additional preferred natural receptor activating compounds and ligands of the invention that are adapted to modulate dopamine secretion via induced receptor activity, include, without limitation, (−)-englerin A, cyclohexyl salicylate (CHS), sandacanol, brazzein, monellin, xylitol, sorbitol, trimethylamine (TMA) and N,N-dimethylethylamine.

In a preferred embodiment, the natural receptor activating compounds and ligands of the invention comprise a molecular weight less than approximately 500.0 Da, whereby the natural receptor activating compounds and ligands can cross the blood-brain-barrier.

According to the invention, the natural receptor activating compounds and ligands of the invention (and, hence, compositions of the invention formed therewith) are adapted to bind to and activate one or more of the following receptors: adipose olfactory receptors, adrenal olfactory receptors, central nervous system (CNS) olfactory receptors, dopaminergic neuron olfactory receptors, mammary olfactory receptors, cardiovascular olfactory receptors, renal olfactory receptors, hepatic olfactory receptors, lymphatic olfactory receptors, ovarian olfactory receptors, prostate olfactory receptors, dermal olfactory receptors, testicular olfactory receptors, hematologic olfactory receptors, trace amine-associated receptors, gastrointestinal (GI) olfactory receptors, free fatty acid receptors, and transient receptor potential ion channels.

In some embodiments of the invention, the natural receptor activating compounds and ligands of the invention referenced above and, hence, compositions of the invention formed therewith are adapted to bind to and activate combinations of the aforementioned receptors, i.e., multiple receptors.

As discussed in detail herein, in a preferred embodiment, the natural receptor activating compounds and ligands of the invention (and, hence, compositions of the invention formed therewith) are adapted to bind to and activate at least one of the following receptors: olfactory receptor family 51 subfamily E member 1 (OR51E1), olfactory receptor family 51 subfamily E member 2 (OR51E2), olfactory receptor family 1 subfamily A member 1 (OR1A1), olfactory receptor family 2 subfamily A member 4 (OR2A4), olfactory receptor family 2subfamily C member 1 (OR2C1), olfactory receptor family 10 subfamily J member 5 (OR10J5), free fatty acid receptor 1 (FFAR1), free fatty acid receptor 4 (FFAR4), olfactory receptor family 2 subfamily W member 1 (OR2W1), olfactory receptor family 2 subfamily B member 11 (OR2B11), olfactory receptor family 2 subfamily J member 3 (OR2J3), transient receptor potential cation channel subfamily A, member 1 (TRPA1), short transient receptor potential channel 4 (TRPC4), taste 1 receptor member 3 (TAS1R3), and trace amine-associated receptor 5 (TAAR5).

As set forth in Applicant's priority U.S. application Ser. No. 18/430,796 and discussed further below, when one of the natural receptor activating compounds and ligands referenced above (and composition formed therewith) binds to and activates olfactory receptor OR51E1,olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor 4 (FFAR4) and/or transient receptor TRPA1, GLP-1 and/or peptide Y-Y (PYY) and/or GIP secretion is induced and, thereby, insulin secretion is modulated in vivo.

Olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor 4 (FFAR4) and transient receptor TRPA1 are thus also referred to herein as “insulin modulating receptors.”

The noted natural receptor activating compounds and ligands, which are adapted to induce GLP-1 and/or PYY secretion and, hence, modulate insulin secretion in vivo via activation of olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor (FFAR4) and transient receptor TRPA1, are thus referred to herein after as “natural insulin modulating compounds and ligands.”

As also discussed in detail below, when one of the natural receptor activating compounds and ligands referenced above (and composition formed therewith) binds to and activates olfactory receptor OR51E2, olfactory receptor OR2A4, transient receptor TRPC4, taste receptor TAS1R3 and/or trace amine-associated receptor TAAR5, dopamine synthesis is induced and, thereby, dopamine secretion is also modulated in vivo.

Olfactory receptor OR51E2, olfactory receptor OR2A4, transient receptor TRPC4, taste receptor TAS1R3 and trace amine-associated receptor TAAR5 are thus also referred to herein as “dopamine modulating receptors.”

The noted natural receptor activating compounds and ligands, which are adapted to induce dopamine synthesis and, thereby, dopamine secretion in vivo via activation of olfactory receptor OR51E2, olfactory receptor OR2A4, transient receptor TRPC4, taste receptor TAS1R3 and/or trace amine-associated receptor TAAR5, are thus also referred to herein as “natural dopamine modulating compounds and ligands.”

As discussed in detail below, olfactory receptor OR51E1 is also capable of inducing dopamine synthesis and, thereby, dopamine secretion modulated in vivo when one of the natural dopamine modulating compounds and ligands referenced above (and composition formed therewith) binds to and activates olfactory receptor OR51E1.

Thus, in some aspects of the invention, olfactory receptor OR51E1 will also be referred to as a “dopamine modulating receptor.”

### Olfactory Receptor-Mediated GLP-1 and PYY Secretion

As indicated above, when one of the natural insulin modulating compounds and ligands referenced above (and composition formed therewith) binds to and activates at least olfactory receptor OR51E1, olfactory receptor OR1A1, olfactory receptor OR2C1 or olfactory receptor OR10J5, GLP-1 and/or PYY secretion is induced in vivo.

The compositions formed from the noted natural insulin modulating compounds and ligands are thus referred to herein as “GLP-1/PYY secretion compositions” (and, in some instances “GLP-1/GIP secretion compositions”).

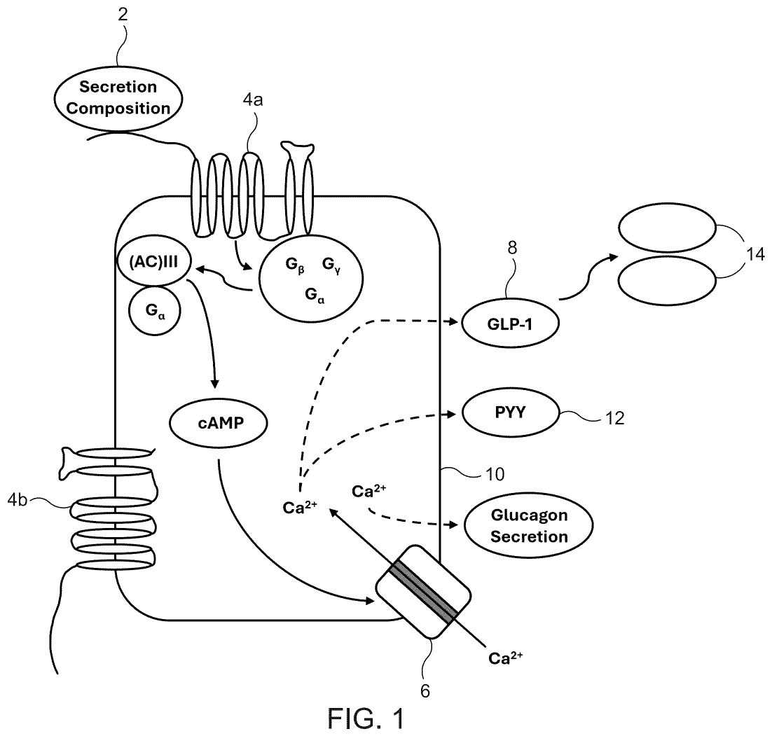

Referring to FIG. 1, according to the invention, when a GLP-1/PYY secretion composition (and, in some instances, a GLP-1/GIP secretion composition) of the invention (denoted “2”) is delivered to a subject or patient, the GLP-1/PYY secretion composition 2 binds to at least one of the ectopic olfactory receptors (ORs) referenced above (denoted “4a, 4b”) disposed on enteroendocrine cells and pancreatic α-cells, in this instance, enteroendocrine L-cells, as depicted in FIG. 1, wherein a representative enteroendocrine L-cell is denoted “10”.

As illustrated in FIG. 1, the binding of the GLP-1/PYY secretion composition 2 to at least one of the ORs 4a, 4b activates the ORs 4a, 4b, in this instance 4a, by inducing a conformational change in the molecular structure of OR 4a, which activates intracellular G_{α}/G_{β}/G_{γ} subunits of OR 4a, whereby the G_{α}/G_{β}/G_{γ} subunits act as a guanine nucleotide exchange factor, wherein a guanine diphosphate (GDP) is exchanged for a guanine triphosphate (GTP), which binds to the Ga subunit of OR 4a.

As further illustrated in FIG. 1, the noted binding of the GTP to the G_{α} subunit induces a dissociation of the G_{α}/G_{β}/G_{γ} subunits of OR 4a into a (i) free G_{α} subunit, which binds to adenyl cyclase (AC) III, and a (ii) G_{β}/G_{γ} complex and, thereby, activates seminal downstream cell signaling processes that induce an increase in intracellular cAMP in the enteroendocrine L-cell 10 and, hence, cells.

The noted increase in intracellular cAMP and a glucose-induced membrane depolarization of the enteroendocrine L-cell 10 (and, hence, cells) opens the voltage-dependent Ca²⁺ (VDC) channels (denoted “6”) of the enteroendocrine L-cells 10, and the resulting Ca²⁺ influx triggers vesicular exocytosis and increases secretion of GLP-1 (denoted “8”) from the enteroendocrine L-cells 10 (and, in some instances, α-cells). In some instances, e.g., when at least one of the activated ORs comprise OR51E1, the resulting Ca²⁺ influx triggers vesicular exocytosis and also increases secretion of PYY (denoted “12”) from the enteroendocrine L-cells 10.

As further illustrated in FIG. 1, the secreted GLP-1 8 binds to and activates GLP-1 receptor proteins on pancreatic β-cells (denoted “14”), which, as indicated above, induces secretion of insulin therefrom.

As indicated above, the GLP-1/PYY secretion compositions of the invention comprise at least one natural insulin modulating compound or ligand, which is specifically adapted to bind to and activate at least olfactory receptor OR51E1, olfactory receptor OR1A1, olfactory receptor OR2C1 or olfactory receptor OR10J5, whereby GLP-1 and/or PYY secretion is induced in vivo.

As discussed below, in some embodiments, the preferred natural insulin modulating compounds and ligands of the GLP-1/PYY secretion compositions, which are adapted to activate at least one of the insulin modulating receptors of the invention; particularly, olfactory receptor OR51E1, olfactory receptor OR1A1, olfactory receptor OR2C1 or olfactory receptor OR10J5, comprise 3-methylpentanoic acid, 4-methylpentanoic acid, farnesol, eugenol, nonanoic acid, pentanol, butyl butyryl lactate, isovaleric acid, geraniol, citronellol, 3-methyl-2,4-nonanedione, estragole, neroli, heptanol, octanol, helional, nonanal, hydroxycitronellal, citral, octanoic acid, musk ketone, (+)-dihydrocarvone, α-cedrene, lyral and thujopsene.

### Olfactory Receptor-Mediated Dopamine Secretion

As indicated above, according to the invention, when one of the natural dopamine modulating compounds and ligands referenced above (and composition formed therewith) binds to and activates at least one of the aforementioned dopamine modulating receptors, i.e., olfactory receptor OR2A4, olfactory receptor OR51E1, olfactory receptor OR51E2, transient receptor TRPC4, taste receptor TASIR3 or trace amine receptor TAAR5, dopamine synthesis is induced and, thereby, dopamine secretion is modulated in vivo.

According to the invention, when a GLP-1/PYY secretion composition (and, in some instances, a GLP-1/GIP secretion composition) comprising one of the natural dopamine modulating compounds and ligands of the invention is delivered to a subject or patient, the natural dopamine modulating compound or ligand, and, hence, GLP-1/PYY secretion composition binds to at least one of the dopamine modulating receptors referenced above, which is disposed on dopaminergic neurons.

The dopaminergic neurons thereafter synthesize dopamine by chemically modifying intracellular tyrosine via the addition of a hydroxyl group, which converts the tyrosine to L-DOPA and subsequently removes a carboxylic acid group from the ethyl side chain linked to the amine group of L-DOPA to yield 3-hydroxytyramine, i.e., dopamine.

The synthesized dopamine is then packaged into synaptic vesicles by vesicular monoamine transporter 2 (VMAT2) and stored in the vesicles until action potentials, e.g., action potentials comprising electrochemical gradients generated by vesicular H⁺-ATPases, induce the secretion of the dopamine from the dopaminergic neurons into the extracellular synaptic cleft between neurons for neurotransmission therebetween.

The secreted dopamine then induces activation of dopaminergic cell signaling pathways by binding to at least one dopamine receptor including, but not limited to, D₁, D₂, D₃, D₄ and D₅ receptors, and at least one ligand-gated ion channel including, but not limited to, GABAA-gated channels, AMPA channels and NMDA channels.

As discussed below, in some embodiments, the preferred natural dopamine modulating compounds and ligands of the GLP-1/PYY secretion compositions, which are adapted to activate at least one of the dopamine modulating receptors of the invention, comprise cyclohexyl salicylate (CHS), eugenol, farnesol, 3-methylpentanoic acid, 4-methylpentanoic acid, butyl butyryl lactate, nonanoic acid, isovaleric acid, acetate, β-ionone, propionic acid, (−)-englerin A, sandacanol, brazzein, monellin, xylitol, sorbitol, trimethylamine (TMA), and N,N-dimethylethylamine.

According to the invention, the EC50 values of the noted natural insulin and dopamine modulating compounds and ligands contained in a GLP-1/PYY secretion composition of the invention (and GLP-1/GIP secretion compositions of the invention, discussed below) can comprise any EC₅₀ values or EC₅₀ value ranges in the range of approximately 1.0 nM to approximately 200.0 mM.

Thus, according to the invention, the EC₅₀ values of natural insulin and dopamine modulating compounds and ligands contained in the GLP-1/PYY secretion compositions of the invention (and GLP-1/GIP secretion compositions of the invention) can comprise in the range of approximately 0.001 μM to approximately 100000.0 μM, approximately 0.002 μM to approximately 10000.0 μM, approximately 0.003 μM to approximately 1000.0 μM, approximately 0.005 μM to approximately 750.0 μM, approximately 0.01 μM to approximately 500.0 μM, approximately 0.05 μM to approximately 50.0 μM, approximately 0.05 μM to approximately 100.0 μM, approximately 0.05 μM to approximately 150.0 μM, approximately 0.05 μM to approximately 200.0 μM, approximately 0.05 μM to approximately 250.0 μM, approximately 0.05 μM to approximately 300.0 μM, approximately 0.05 μM to approximately 350.0 μM, approximately 0.05 μM to approximately 400.0 μM, approximately 0.05 μM to approximately 450.0 μM, approximately 0.05 μM to approximately 500.0 μM, approximately 0.1 μM to approximately 50.0 μM, approximately 0.1 μM to approximately 100.0 μM, approximately 0.1 μM to approximately 150.0 μM, approximately 0.1 μM to approximately 200.0 μM, approximately 0.1 μM to approximately 250.0 μM, approximately 0.1 μM to approximately 300.0 μM, approximately 0.1 μM to approximately 350.0 μM, approximately 0.1 μM to approximately 400.0 μM, approximately 0.1 μM to approximately 450.0 μM, approximately 0.1 μM to approximately 500.0 μM, approximately 0.1 μM to approximately 1000.0 μM, approximately 0.1 μM to approximately 1500.0 μM, approximately 0.1 μM to approximately 2000.0 μM, approximately 0.1 μM to approximately 2500.0 μM, approximately 0.1 μM to approximately 3000.0 μM, approximately 0.25 μM to approximately 50.0 μM, approximately 0.25 μM to approximately 100.0 μM, approximately 0.25 μM to approximately 150.0 μM, approximately 0.25 μM to approximately 200.0 μM, approximately 0.25 μM to approximately 250.0 μM, approximately 0.25 μM to approximately 300.0 μM, approximately 0.25 μM to approximately 350.0 μM, approximately 0.25 μM to approximately 400.0 μM, approximately 0.25 μM to approximately 450.0 μM, approximately 0.25 μM to approximately 500.0 μM, approximately 0.5 μM to approximately 300.0 μM, approximately 1.0 μM to approximately 50.0 μM, approximately 1.0 μM to approximately 100.0 μM, approximately 1.0 μM to approximately 150.0 μM, approximately 1.0 μM to approximately 200.0 μM, approximately 1.0 μM to approximately 250.0 μM, approximately 1.0 μM to approximately 300.0 μM, approximately 1.0 μM to approximately 350.0 μM, approximately 1.0 μM to approximately 400.0 μM, approximately 1.0 μM to approximately 450.0 μM, approximately 1.0 μM to approximately 500.0 μM, approximately 2.5 μM to approximately 100.0 μM, approximately 5.0 μM to approximately 75.0 μM, approximately 7.5 μM to approximately 50.0 μM, approximately 10.0 μM to approximately 25.0 μM, and/or any EC₅₀ values therebetween.

According to the invention, the EC₅₀ values of natural insulin and dopamine modulating compounds and ligands contained in the GLP-1/PYY secretion compositions of the invention (and GLP-1/GIP secretion compositions of the invention) can also comprise in the range of approximately 0.001 μM to approximately 10.0 μM, approximately 0.005 μM to approximately 7.5 μM, approximately 0.01 μM to approximately 5.0 μM, approximately 0.03 μM to approximately 2.5 μM, approximately 0.05 μM to approximately 1.5 μM, approximately 0.03 μM to approximately 1.0 μM, approximately 0.1 μM to approximately 0.5 μM, and/or any EC₅₀ values therebetween.

In some embodiments, the EC₅₀ values of natural insulin and dopamine modulating compounds and ligands contained in the GLP-1/PYY secretion compositions of the invention (and GLP-1/GIP secretion compositions of the invention) comprise at least 0.001 μM, at least 0.002 μM, at least 0.003 μM, at least 0.004 μM, at least 0.005 μM, at least 0.006 μM, at least 0.007 μM, at least 0.008 μM, at least 0.009 μM, at least 0.01 μM, at least 0.02 μM, at least 0.03 μM, at least 0.04 μM, at least 0.05 μM, at least 0.06 μM, at least 0.07 μM, at least 0.08 μM, at least 0.09 μM, at least 0.1 μM, at least 0.2 μM, at least 0.3 μM, at least 0.4 μM, at least 0.5 μM, at least 0.6 μM, at least 0.7 μM, at least 0.8 μM, at least 0.9 μM, at least 1.0 μM, at least 2.0 μM, at least 3.0 μM, at least 4.0 μM, at least 5.0 μM, at least 6.0 μM, at least 7.0 μM, at least 8.0 μM, at least 9.0 μM, at least 10.0 μM, at least 20.0 μM, at least 30.0 μM, at least 40.0 μM, at least 50.0 μM, at least 60.0 μM, at least 70.0 μM, at least 80.0 μM, at least 90.0 μM, at least 100.0 μM, at least 200.0 μM, at least 300.0 μM, at least 400.0 μM, at least 500.0 μM, at least 600.0 μM, at least 700.0 μM, at least 800.0 μM, at least 900.0 μM, or at least 1,000.0 μM.

In a preferred embodiment, the GLP-1/PYY secretion compositions of the invention comprise at least one of the natural insulin modulating compounds and ligands referenced above that is adapted to bind to and activate at least one of the following insulin modulating receptors: olfactory receptor OR51E1, olfactory receptor OR1A1, olfactory receptor OR2C1 and olfactory receptor OR10J5.

As indicated above, in some embodiments, the GLP-1/PYY secretion compositions of the invention also comprise at least one natural dopamine modulating compound or ligand referenced above that is adapted to bind to and activate at least one of the aforementioned dopamine modulating receptors, i.e., olfactory receptor OR2A4, olfactory receptor OR51E1, olfactory receptor OR51E2, transient receptor TRPC4, taste receptor TAS1R3 or trace amine receptor TAAR5.

The pharmacodynamic activity induced via activation of each of the receptors is discussed in detail below.

### Olfactory Receptor OR51E1 Modulation

In a preferred embodiment of the invention, the GLP-1/PYY secretion compositions of the invention (and GLP-1/GIP secretion compositions of the invention, discussed below) comprise at least one natural insulin modulating compound or ligand, which is specifically adapted to activate and, hence, modulate olfactory receptor OR51E1 activity.

As indicated above, activation of olfactory receptor OR51E1 induces a glucose-induced membrane depolarization of endogenous GI cells, more particularly, L-enteroendocrine (and, in some instances, pancreatic α-cells) and, thereby, opens the voltage-dependent Ca²⁺ (VDC) channels of the L-cells, whereby the resulting Ca²⁺ influx triggers vesicular exocytosis and increases secretion of GLP-1 and PYY from the cells.

The secreted GLP-1 binds to and activates GLP-1 receptor proteins on pancreatic β-cells, which induces secretion of insulin.

In a preferred embodiment, activation of olfactory receptor OR51E1 also effectuates secretion modulation and/or activation of additional endocrine factors, including, without limitation, ghrelin, gastrin, cholecystokinin (CCK), bombesin/gastrin releasing peptide (BBS/GRP), neurotensin (NT), glucagon-like peptide 2 (GLP-2), calcitonin gene-related peptide (CGRP), chromogranin A, enteroglucagon, galanin, leptin, motilin, amylin, neuropeptide Y (NPY), pancreatic polypeptide, substance P, oxyntomodulin, and somatostatin.

As indicated above, olfactory receptor OR51E1 is also capable of inducing dopamine synthesis and, thereby, dopamine secretion modulation in vivo when one of the natural dopamine modulating compounds and ligands referenced above (and composition formed therewith) binds to and activates olfactory receptor OR51E1.

Indeed, as discussed in detail below, when one of the natural dopamine modulating compounds and ligands referenced above (and composition formed therewith) binds to and activates olfactory receptor OR51E1, the natural dopamine modulating compound or ligand induces dopaminergic activation of olfactory receptor OR51E1 and, thereby, induced dopamine synthesis by dopaminergic neurons.

In some embodiments of the invention, the preferred natural insulin modulating compounds and ligands of the GLP-1/PYY secretion compositions (and GLP-1/GIP secretion compositions) of the invention, which are specifically adapted to activate olfactory receptor OR51E1 in vivo, comprise 3-methylpentanoic acid, 4-methylpentanoic acid, farnesol, eugenol, nonanoic acid, pentanol, butyl butyryl lactate and isovaleric acid.

### Olfactory Receptor OR1A1 Modulation

In some embodiments, the GLP-1/PYY secretion compositions (and GLP-1/GIP secretion compositions) of the invention comprise one or more natural insulin modulating compounds and ligands that are specifically adapted to activate and, hence, modulate olfactory receptor OR1A1 activity, whereby pharmacodynamic activity similar to that induced via activation of olfactory receptor OR51E1 by a natural insulin modulating compound or ligand (discussed above) is induced.

In some embodiments of the invention, the preferred natural insulin modulating compounds and ligands of the GLP-1/PYY secretion compositions (and GLP-1/GIP secretion compositions) of the invention, which are specifically adapted to activate olfactory receptor OR1A1 in vivo, comprise geraniol, citronellol, 3-methyl-2,4-nonanedione, estragole, neroli, heptanol, octanol, helional, nonanal, hydroxycitronellal and citral.

### Olfactory Receptor OR2C1 Modulation

In some embodiments, the GLP-1/PYY secretion compositions (and GLP-1/GIP secretion compositions) of the invention comprise one or more natural insulin modulating compounds and ligands that are specifically adapted to activate and, hence, modulate olfactory receptor OR2C1 activity, whereby the following pharmacodynamic activity is induced.

Activation of olfactory receptor OR2C1 induces Ca²⁺ release from the endoplasmic reticulum of pancreatic β-cells through the phospholipase C-inositol triphosphate-dependent (PLC-IP3) pathway and, thereby, an increased concentration of intracellular Ca²⁺. The increase in intracellular Ca²⁺ then activates the CaMKK/CaMKIV pathway, which induces seminal insulinogenic processes, including glucokinase (GK) expression, and thereby glucose absorption by endogenous cells and glucose-stimulated insulin secretion (GSIS) from pancreatic islet cells.

The olfactory receptor OR2C1 thus indirectly induces GLP-1 production and secretion by endogenous GI cells via the above noted insulinogenic processes, which facilitate glucose-induced membrane depolarization of the GI cells induced by olfactory receptor activation (e.g., olfactory receptor OR51E1 activation) and, hence, secretion of GLP-1 from the GI cells resulting therefrom.

Activation of olfactory receptor OR2C1 similarly effectuates secretion modulation and/or activation of at least one of the aforementioned additional endocrine factors.

In some embodiments of the invention, the preferred natural insulin modulating compounds and ligands of the GLP-1/PYY secretion compositions (and GLP-1/GIP secretion compositions) of the invention, which are specifically adapted to activate olfactory receptor OR2C1 in vivo, comprise octanoic acid, eugenol, musk ketone and (+)-dihydrocarvone.

### Olfactory Receptor OR10J5 Modulation

In some embodiments of the invention, the GLP-1/PYY secretion compositions (and GLP-1/GIP secretion compositions) of the invention comprise one or more natural insulin modulating compounds and ligands that are specifically adapted to activate and, hence, modulate olfactory receptor OR10J5 activity, whereby the following pharmacodynamic activity is induced.

Activation of olfactory receptor OR10J5 induces downregulation of the seminal lipogenesis associated gene expression, including the expression of C/EBPα, PPARγ, RXR, LXRα, SREBP-1c, ap2, FAS, SCDI, ACC, and mtGPAT genes, and upregulation of mitochondrial and thermogenic gene expression, including the expression of PGC-1α, PRDM16, UCP1, Cytc, Cox4, and Cidea genes through the cAMP/PKA/HSL pathway.

The above noted downregulation of the seminal lipogenesis associated gene expression and upregulation of mitochondrial and thermogenic gene expression modulates lipid metabolism by inhibiting lipogenesis and, thus, reducing lipid accumulation in hepatic cells.

The olfactory receptor OR10J5 also indirectly induces GLP-1 production and secretion by endogenous GI cells via the above noted downregulation of the seminal lipogenesis associated gene expression, which restores normal lipogenic metabolic function and, thereby, facilitates glucose-induced membrane depolarization of the GI cells induced by olfactory receptor activation (e.g., olfactory receptor OR51E1 activation) and, hence, secretion of GLP-1 from the GI cells resulting therefrom.

In a preferred embodiment, the activation of olfactory receptor OR10J5 similarly effectuates secretion modulation and/or activation of at least one of the aforementioned additional endocrine factors.

In some embodiments of the invention, the preferred natural insulin modulating compounds and ligands of the GLP-1/PYY secretion compositions (and GLP-1/GIP secretion compositions) of the invention, which are specifically adapted to activate olfactory receptor OR10J5 in vivo, similarly comprise α-cedrene, lyral and thujopsene.

### Olfactory Receptor-Mediated and Free Fatty Acid Receptor-Mediated (GIP) Secretion

In some embodiments of the invention, the natural insulin modulating compounds and ligands of the invention, and compositions of the invention formed therewith, are specifically adapted to bind to and activate at least at least one receptor that induces GIP secretion in vivo including, without limitation, free fatty acid receptor FFAR1, free fatty acid receptor FFAR4, olfactory receptor OR2W1, olfactory receptor OR2B11, olfactory receptor OR2J3, and transient receptor TRPA1, whereby, as discussed in detail below, GIP secretion is induced in vivo.

The compositions formed from the noted natural insulin modulating compounds and ligands are thus referred to herein as “GIP secretion compositions” (and, in some instances “GLP-1/GIP secretion compositions”).

According to the invention, the GIP secretion compositions of the invention (and GLP-1/GIP secretion compositions of the invention, discussed below), when delivered to a patient or subject, effectuate the following highly effective and, hence, desirable pharmacodynamic activity.

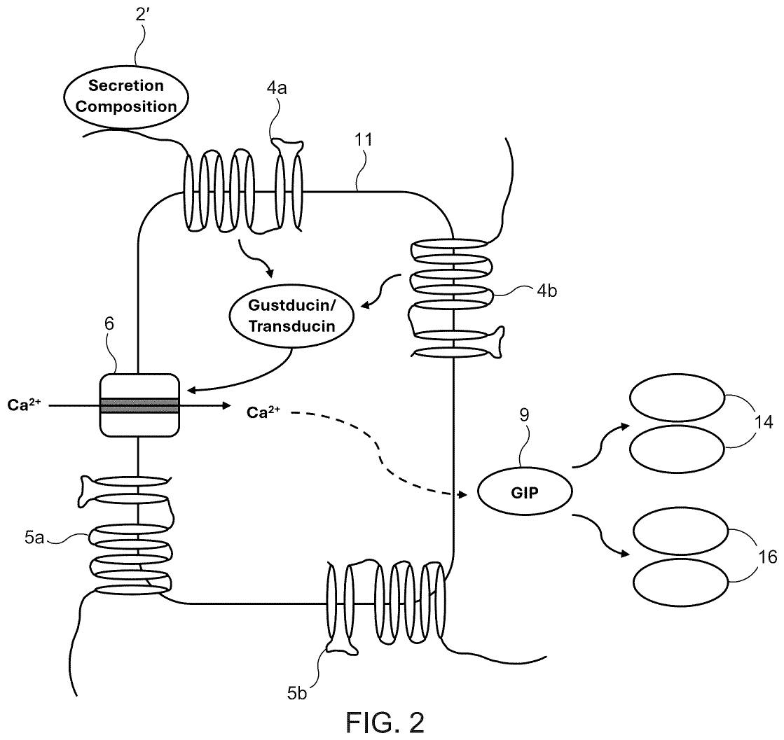

Referring to FIG. 2, when a GIP secretion composition of the invention (and GLP-1/GIP secretion composition of the invention) (denoted “2”) is delivered to a subject or patient, the GIP secretion composition 2′ binds to at least one of the ectopic olfactory receptors referenced above (denoted “4a, 4b”) and at least one of the free fatty acid receptors (denoted “5a, 5b”) disposed on endogenous cells, in this instance, enteroendocrine K-cells, as depicted in FIG. 2, wherein a representative enteroendocrine K-cell is denoted “11”, whereby a gustducin-mediated cell signaling pathway is activated, which induces membrane depolarization of the enteroendocrine K-cell 11 (and, hence, cells) and opens the voltage-dependent Ca²⁺ (VDC) channels (denoted “6”) of the enteroendocrine K-cell(s) 11, wherein the resulting Ca²⁺ influx induces vesicular exocytosis and increased secretion of GIP (denoted “9”) from the enteroendocrine K-cell(s) 11.

As illustrated in FIG. 2, the secreted GIP 9 binds to and activates GIP receptor proteins on pancreatic β-cells (denoted “14”), which, induces secretion of insulin.

As further illustrated in FIG. 2, the secreted GIP 9 also binds to and activates GIP receptor proteins on endogenous GI cells (denoted “16”), such as islet cells of the pancreas, to promote pancreatic β-cell survival and prevent apoptosis of pancreatic β-cells by activating the CAMP response element-binding (CREB) and Akt/PKB pathways, thus, directly, and indirectly maintaining a stable population of insulin-producing pancreatic β-cells in vivo.

As indicated above, the GIP secretion compositions (and GLP-1/GIP secretion compositions) of the invention comprise at least one receptor activating compound or ligand that is specifically adapted to bind to and activate at least free fatty acid receptor FFAR1, free fatty acid receptor FFAR4, olfactory receptor OR2W1, olfactory receptor OR2B11, olfactory receptor OR2J3 or transient receptor TRPA1.

As discussed below, in some embodiments, the preferred natural insulin modulating compounds and ligands of the GIP secretion compositions (and GLP-1/GIP secretion compositions) comprise a medium-chain free fatty acid, including, without limitation, lauric acid, caproic acid, caprylic acid and capric acid; a long-chain free fatty acid, including, without limitation, palmitic acid and stearic acid; an omega-3 polyunsaturated acid, including, without limitation, alpha-linoleic acid, docosahexaenoic acid, and eicosatetraenoic acid; 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione, cis-3-hexen-1-ol, 2-ethyl-3,5-dimethylpyrazine, coumarin, dicyclohexyl disulfide, spearmint, coffee difuran, quinoline, cinnamaldehyde, allyl isothiocyanate, farnesyl thiosalicylic acid, formalin, hydrogen peroxide, 4-hydroxynonenal and acrolein.

According to the invention, the EC₅₀ values of the noted natural insulin modulating compounds and ligands contained in a GIP secretion composition of the invention (and GLP-1/GIP secretion compositions of the invention) can similarly comprise any of the EC₅₀ values or EC₅₀ value ranges set forth above.

As indicated above, the natural insulin modulating compounds and ligands of the GIP secretion compositions are specifically adapted to bind to and activate at least free fatty acid receptor FFAR1, free fatty acid receptor FFAR4, olfactory receptor OR2W1, olfactory receptor OR2B11, olfactory receptor OR2J3 or transient receptor TRPA1.

The pharmacodynamic activity induced via activation of free fatty acid receptor FFAR1, free fatty acid receptor FFAR4, olfactory receptor OR2W1, olfactory receptor OR2B11, olfactory receptor OR2J3 and transient receptor TRPA1 is discussed in detail below.

### Fatty Acid Receptor FFAR1 and FFAR4 Modulation

In some embodiments, the GIP secretion compositions of the invention (and GLP-1/GIP secretion compositions of the invention) comprise one or more natural insulin modulating compounds and ligands that are specifically adapted to activate and, hence, modulate free fatty acid receptor FFAR1 and/or free fatty acid receptor FFAR4 activity, whereby the following pharmacodynamic activity is induced.

As indicated above, activation of free fatty acid receptors FFAR1 and FFAR4 induces membrane depolarization of enteroendocrine cells and opens voltage-dependent Ca²⁺ (VDC) channels of the enteroendocrine cells, wherein the resulting Ca²⁺ influx induces increased secretion of GIP from the enteroendocrine cells.

Activation of free fatty acid receptor FFAR1 and/or FFAR4, can, in some instances, also induce activation of G_{aq/11} and β-arrestin signaling pathways and, thereby, stimulate further GIP secretion.

As also indicated, the secreted GIP binds to and activates GIP receptor proteins on pancreatic β-cells, which induces secretion of insulin.

The secreted GIP also binds to and activates GIP receptor proteins on endogenous GI cells, such as islet cells of the pancreas, to promote pancreatic β-cell survival and prevent apoptosis of pancreatic β-cells by activating the CAMP response element-binding (CREB) and Akt/PKB pathways, thus, directly, and indirectly maintaining a stable population of insulin-producing pancreatic β-cells in vivo.

In a preferred embodiment, activation of free fatty acid receptor FFAR1 and/or FFAR4, also effectuates secretion modulation and/or activation of at least one of the aforementioned additional endocrine factors.

In some embodiments of the invention, the natural insulin modulating compounds and ligands of the GIP secretion compositions (and GLP-1/GIP secretion compositions) of the invention, which are specifically adapted to activate free fatty acid receptor FFAR1 and/or FFAR4 in vivo, comprise a medium-chain free fatty acid, a long-chain free fatty acid, and an omega-3 polyunsaturated fatty acid.

In some embodiments, the natural insulin modulating compounds and ligands of the GIP secretion compositions (and GLP-1/GIP secretion compositions) of the invention thus comprise lauric acid, caproic acid, caprylic acid, capric acid, palmitic acid, stearic acid, alpha-linoleic acid, docosahexaenoic acid and eicosatetraenoic acid.

### Olfactory Receptor OR2W1 Modulation

In some embodiments of the invention, the GIP secretion compositions (and GLP-1/GIP secretion compositions) of the invention comprise one or more natural insulin modulating compounds and ligands that are specifically adapted to activate and, hence, modulate olfactory receptor OR2W1 activity, whereby the following pharmacodynamic activity is induced.

Activation of olfactory receptor OR2W1 by natural insulin modulating compound or ligand induces a conformational change in the molecular structure of olfactory receptor OR2W1, which activates intracellular G_{α}/G_{β}/G_{γ} subunits of the receptor (See FIG. 1), whereby the subunits act as a guanine nucleotide exchange factor, wherein a guanine diphosphate (GDP) is exchanged for a guanine triphosphate (GTP), which binds to the G_{α} subunit of olfactory receptor OR2W1.

The noted binding of the GTP to the G_{α} subunit induces a dissociation of the G_{α}/G_{β}/G_{γ} subunits of olfactory receptor OR2W1 into a (i) free Gα subunit, which binds to adenyl cyclase (AC) III (See FIG. 1), and a (ii) G_{β}/G_{γ} complex (See also FIG. 1) and, thereby, activates seminal downstream cell signaling processes that induce an increase in intracellular cAMP in endogenous cells, such as enteroendocrine cells.

As depicted in FIG. 2, by virtue of the intracellular cAMP level increase, among other cell signaling processes, opening of cyclic nucleotide gated Ca²⁺ channels is induced, which results in increased cellular Ca²⁺ and, thereby, increased induction of secretion of GIP from the endogenous cells.

The secreted similarly GIP binds to and activates GIP receptor proteins on pancreatic β-cells, which, as indicated above, (i) induces secretion of insulin, (ii) promotes pancreatic β-cell survival, and (iii) prevents apoptosis of pancreatic β-cells by activating the cAMP response element-binding (CREB) and Akt/PKB pathways, thus, directly and indirectly maintaining a stable population of insulin-producing pancreatic β-cells in vivo.

In some embodiments, the activation of olfactory receptor OR2W1 similarly effectuates secretion modulation and/or activation of at least one of the aforementioned additional endocrine factors.

In some embodiments of the invention, the preferred natural insulin modulating compounds and ligands of the GIP secretion compositions (and GLP-1/GIP secretion compositions), which are adapted to activate OR2W1 in vivo comprise 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione, and cis-3-hexen-1-ol.

### Olfactory Receptor OR2B11 Modulation

In some embodiments of the invention, the GIP secretion compositions (and GLP-1/GIP secretion compositions) of the invention comprise one or more natural insulin modulating compounds and ligands that are specifically adapted to activate and, hence, modulate olfactory receptor OR2B11 activity, whereby pharmacodynamic activity similar to that induced via activation of olfactory receptor OR2W1 (discussed above) is induced.

In some embodiments of the invention, the preferred natural insulin modulating compounds and ligands of the GIP secretion compositions (and GLP-1/GIP secretion compositions) of the invention, which are adapted to activate OR2B11 in vivo comprise 2-ethyl-3,5-dimethylpyrazine, coumarin, dicyclohexyl disulfide, spearmint, coffee difuran, quinoline, and cinnamaldehyde.

### Olfactory Receptor OR2J3 Modulation

In some embodiments of the invention, the GIP secretion compositions of the invention (and GLP-1/GIP secretion compositions of the invention) comprise one or more natural insulin modulating compounds and ligands that are specifically adapted to activate and, hence, modulate olfactory receptor OR2J3 activity, whereby pharmacodynamic activity similar to that induced via activation of olfactory receptor OR2W1 (discussed above) is induced.

In some embodiments of the invention, the preferred natural insulin modulating compounds and ligands of the GIP secretion compositions (and GLP-1/GIP secretion compositions), which are adapted to activate, which are adapted to activate OR2J3 in vivo comprise cis-3-hexen-1-ol and cinnamaldehyde.

### Transient Receptor TRPA1 Modulation

In some embodiments of the invention, the GIP secretion compositions (and GLP-1/GIP secretion compositions) of the invention comprise one or more natural insulin modulating compounds and ligands that are specifically adapted to activate and, hence, modulate transient receptor TRPA1 activity, whereby the following pharmacodynamic activity is induced.

It is believed that, when transient receptor TRPA1 is activated, the compound cellular Ca²⁺ is increased, whereby serotonin (5-HT) secretion from endogenous enterochromaffin cells is increased.

The serotonin secreted from the enterochromaffin cells binds to 5-HT receptors of endogenous gastrointestinal cells and, thereby, induces GIP secretion by the endogenous cells.

In some embodiments of the invention, the preferred natural insulin modulating compounds and ligands of the GIP secretion compositions (and GLP-1/GIP secretion compositions) of the invention, which are adapted to activate transient receptor TRPA1 in vivo, comprise allyl isothiocyanate, cinnamaldehyde, farnesyl thiosalicylic acid, formalin, hydrogen peroxide, 4-hydroxynonenal, and acrolein.

In some embodiments, the GIP secretion compositions (and GLP-1/GIP secretion compositions) of the invention are formulated and adapted to activate and, hence, modulate at least one olfactory receptor; specifically, olfactory receptor OR51E1, olfactory receptor OR2C1 or olfactory receptor OR2W1, and at least one free fatty acid receptor; specifically, free fatty acid receptor FFAR1 or free fatty acid receptor FFAR4, and, in some instances, at least one transient receptor; specifically, transient receptor TRPA1 in vivo when delivered to a patient.

In a preferred embodiment, the GIP secretion compositions of the invention and, as discussed below, GLP-1/GIP secretion compositions of the invention, are adapted to induce at least 50% activation of at least olfactory receptor OR2W1 and/or olfactory receptor OR2B11 and/or OR2J3, free fatty acid receptor FFAR1 and/or free fatty acid receptor FFAR4, and/or transient receptor TRPA1 in vivo when delivered to a patient.

### Concomitant Modulation of GLP-1 and GIP Secretion

In some embodiments of the invention, the natural insulin modulating compounds and ligands, and compositions of the invention formed therewith, are specifically adapted to bind to and activate and, hence, modulate at least one insulin modulating receptor that induces GLP-1 secretion and at least one insulin modulating receptor that induces GIP secretion in vivo, including, without limitation, olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor FFAR4 and transient receptor TRPA1, whereby the pharmacodynamic activity associated with the selective insulin modulating receptor described above is induced.

The compositions formed from the noted natural receptor activating compounds and ligands are thus referred to herein as “GLP-1/GIP secretion compositions.”

According to the invention, GLP-1/GIP secretion compositions can thus comprise one or more of the natural insulin modulating compounds and ligands of the invention referenced above.

In a preferred embodiment, the natural insulin modulating compounds and ligands of the GLP-1/GIP secretion compositions of the invention comprise 3-methylpentanoic acid, 4-methylpentanoic acid, farnesol, eugenol, nonanoic acid, pentanol, butyl butyryl lactate, isovaleric acid, geraniol, citronellol, 3-methyl-2,4-nonanedione, estragole, neroli, heptanol, octanol, helional, nonanal, hydroxycitronellal, citral, octanoic acid, musk ketone, (+)-dihydrocarvone, α-cedrene, lyral, thujopsene, lauric acid, caproic acid, caprylic acid, capric acid, palmitic acid, stearic acid, alpha-linoleic acid, docosahexaenoic acid, eicosatetraenoic acid, 2-heptanone, 1-octanal, (−)-citronellol, hexanal, 3-octanone, hexyl acetate, 1-hexanol, octanoic acid, 1-heptanol, allyl phenylacetate, benzyl acetate, 3,4-hexanedione, cis-3-hexen-1-ol, 2-ethyl-3,5-dimethylpyrazine, coumarin, dicyclohexyl disulfide, spearmint, coffee difuran, quinoline, cinnamaldehyde, allyl isothiocyanate, farnesyl thiosalicylic acid, formalin, hydrogen peroxide, 4-hydroxynonenal and acrolein.

As indicated above, according to the invention, the EC₅₀ value of the noted natural insulin modulating compounds and ligands contained in a GLP-1/GIP secretion composition of the invention can comprise any of the aforementioned EC₅₀ value ranges and EC₅₀ values therebetween.

As indicated above, in a preferred embodiment, the GLP-1/GIP secretion compositions of the invention are formulated and adapted to activate and, hence, modulate at least one of the insulin modulating receptors of the invention, i.e., olfactory receptor OR51E1, olfactory receptor OR1A1, olfactory receptor OR2C1, olfactory receptor OR10J5, olfactory receptor OR2W1, olfactory receptor OR2B11, olfactory receptor OR2J3, free fatty acid receptor FFAR1, free fatty acid receptor (FFAR4) and transient receptor TRPA1.

In some embodiments, the GLP-1/GIP secretion compositions of the invention are specifically formulated and adapted to activate at least olfactory receptor OR51E1 and free fatty acid receptor FFAR1.

In a preferred embodiment, the GLP-1/GIP secretion compositions of the invention are adapted to induce at least 50% activation of at least olfactory receptor OR51E1 and/or olfactory receptor OR1A1 and/or olfactory receptor OR2W1 and/or olfactory receptor OR2B11, free fatty acid receptor FFAR1 and/or free fatty acid receptor FFAR4, and/or transient receptor TRPA1 in vivo when delivered to a patient.

As indicated above, in a preferred embodiment, the GLP-1/GIP secretion compositions of the invention are adapted to induce at least 50% activation of multiple receptors; particularly, olfactory receptor OR51E1 and free fatty acid receptor FFAR1 or free fatty acid receptor FFAR4.

According to the invention, modulating the activity of multiple receptors, e.g., olfactory receptors and/or free fatty acid receptors and/or transient receptor potential ion channels, as described herein, results in elevated endocrine factor levels.

In some embodiments, modulating the activity of multiple receptors, e.g., olfactory receptors and/or free fatty acid receptors and/or transient receptor potential ion channels, as described herein, results in synergistically elevated endocrine factor levels.

In some embodiments, modulating the activity of multiple receptors, e.g., olfactory receptors and/or free fatty acid receptors and/or transient receptor potential ion channels, as described herein, results in elevated endocrine factor secretion.

In some embodiments, modulating the activity of multiple receptors, e.g., olfactory receptors and/or free fatty acid receptors and/or transient receptor potential ion channels, as described herein, results in synergistically elevated endocrine factor secretion.

In some embodiments, modulating the activity of multiple receptors, e.g., olfactory receptors and/or free fatty acid receptors and/or transient receptor potential ion channels, as described herein, results in endocrine factor secretion higher than endocrine factor secretion induced when modulating the activity of any single receptor alone.

In some embodiments, the GLP-1/PYY, GIP and GLP-1/GIP secretion compositions of the invention further comprise a physiologically suitable (or acceptable) carrier (also referred to herein as a physiologically suitable (or acceptable) excipient, or physiologically suitable (or acceptable) excipient selected based on a chosen route of administration, e.g., oral administration, and standard pharmaceutical practice.

According to the invention, suitable aqueous and non-aqueous carriers that can be employed in the secretion compositions of the invention include water, ethanol, polyols (such as glycerol, glycerin-based water, propylene glycol, polyethylene glycol, and the like), and suitable mixtures thereof; vegetable oils, such as olive oil; buffers, such as neutral buffered saline, phosphate buffered saline and the like; carbohydrates, such as glucose, mannose, sucrose, and dextran, mannitol; proteins; polypeptides, and amino acids, such as glycine; antioxidants; chelating agents such as ethylenediaminetetraacetic acid (EDTA) or glutathione; adjuvants (e.g., aluminum hydroxide); and injectable organic esters, such as ethyl oleate and cyclodextrins.

According to the invention, the GLP-1/PYY, GIP and GLP-1/GIP secretion compositions of the invention can be manufactured by means of conventional mixing, dissolving, granulating, dragee-making, levigating, emulsifying, encapsulating, entrapping, and lyophilizing processes. The manufactured compositions can take the form of solutions, suspensions, emulsion, tablets, pills, pellets, capsules, capsules containing liquids, powders, sustained-release formulations, suppositories, emulsions, aerosols, sprays, suspensions, and other forms suitable for administration to a patient.

In some embodiments, proper fluidity of a composition is maintained via coating materials, such as lecithin.

According to the invention, the GLP-1/PYY, GIP and GLP-1/GIP secretion compositions of the invention can be formulated into any known form suitable for parenteral administration, e.g., injection or infusion. Alternatively, the compositions can be formulated for oral administration, nasal or other mucosal tissue administration, or administration as a suppository (e.g., for small molecules). The compositions can also comprise formulation additives, such as suspending agents, preservatives, stabilizers and/or dispersants, and preservation agents.

According to the invention, the GLP-1/PYY, GIP and GLP-1/GIP secretion compositions can thus be administered to a patient via any suitable method, including, without limitation, oral, sublingual, inhalation, intranasal, epidural, intracerebral, transdermal, topical, and injection administration means.

The GLP-1/PYY, GIP and GLP-1/GIP secretion compositions of the invention can also be administered to a patient via intraarterial, subcutaneous, intradermal, intratumoral, intranodal, intramedular, intramuscular, intranasally, and intraperitoneal means.

According to the invention, the GLP-1/PYY, GIP and GLP-1/GIP secretion compositions of the invention can also be incorporated into various ingestible fluids, such as flavored waters and coffee.

According to the invention, the GLP-1/PYY, GIP and GLP-1/GIP secretion compositions of the invention can also be incorporated into a food item, such as a cracker, and/or a nutritional supplement or supplemental food item, such as protein bar.

According to the invention, the GLP-1/PYY, GIP and GLP-1/GIP secretion compositions of the invention can be administered at any of the following dosage ranges: from about 1.0 μg/kg to about 1.0 kg/kg, about 10.0 μg/kg to about 100.0 g/kg, about 10.0 μg/kg to about 25.0 mg/kg, about 100.0 μg/kg to about 50.0 g/kg, about 100.0 μg/kg to about 50.0 mg/kg, about 500.0 μg/kg to about 25.0 g/kg, about 500.0 μg/kg to about 100.0 mg/kg, about 1.0 mg/kg to about 10.0 g/kg, about 1.0 mg/kg to about 50.0 mg/kg, about 5.0 mg/kg to about 5.0 g/kg, about 5.0 mg/kg to about 25.0 mg/kg, about 10.0 mg/kg to about 2.5 g/kg, about 10.0 mg/kg to about 200.0 mg/kg, about 25.0 mg/kg to about 1.5 g/kg, about 25.0 mg/kg to about 750.0 mg/kg, about 50.0 mg/kg to about 1.0 g/kg, about 50.0 mg/kg to about 600.0 mg/kg, about 75.0 mg/kg to about 550.0 mg/kg, about 100.0 mg/kg to about 500.0 mg/kg, about 150.0 mg/kg to about 400.0 mg/kg, and about 200.0 mg/kg to about 350.0 mg/kg.

The GLP-1/PYY, GIP and GLP-1/GIP secretion compositions of the invention can also be administered at any dosage between the above referenced dosage ranges.

The GLP-1/PYY, GIP and GLP-1/GIP secretion compositions of the invention can thus also be administered at a dosage of at least about 1.0 μg/kg, at least about 10.0 μg/kg, at least about 100.0 g/kg, at least about 500.0 μg/kg, at least about 1.0 mg/kg, at least about 5.0 mg/kg, at least about 10.0 mg/kg, at least about 25.0 mg/kg, at least about 50.0 mg/kg, at least about 75.0 mg/kg, at least about 100.0 mg/kg, at least about 150.0 mg/kg, and at least about 200.0 mg/kg.

According to the invention, GLP-1/PYY, GIP and GLP-1/GIP secretion compositions of the invention can also be administered at one of the dosage ranges (and/or dosages therebetween) over a prescribed time, by way of example, from about 1.0 μg to about 1.0 kg per day, from about 100.0 μg to about 500.0 g per day, from about 500.0 μg to about 100.0 g per day, from about 1.0 mg to about 20.0 g per day, from about 2.5 mg to about 15.0 g per day, from about 5.0 mg to about 10.0 g per day, from about 10.0 mg to about 5.0 g per day, from about 25.0 mg to about 2.5 g per day, from about 50.0 mg to about 2.0 g per day, from about 100.0 mg to about 1.5 g per day, from about 150.0 mg to about 1.0 g per day, from about 200.0 mg to about 750.0 mg per day, and from about 250.0 mg to about 500.0 mg per day.

According to the invention, the noted dosages and delivery protocols are sufficient to induce sustained (i.e., extended periods) of GLP-1, PYY and/or GIP secretion in vivo.

In some embodiments, the noted dosages and delivery protocols are also sufficient to induce sustained dopamine synthesis and, thereby, dopamine secretion modulation in vivo.

As indicated above, the GLP-1/PYY, GIP and GLP-1/GIP compositions of the invention, when delivered to a patient, effectively and safely modulate, i.e., increase, insulin and PYY secretion and, hence, can be readily employed to treat neurodegenerative diseases and disorders, as well as a multitude of other physiological disorders associated with (or caused by) abnormal insulin secretion (and insulin resistance); particularly, type 2 diabetes mellitus.

Indeed, as discussed in detail below, recent studies reflect that insulin resistance and, hence, hyperglycemia associated therewith is also associated with Parkinson's disease development and progression through mitochondrial dysfunction, reactive oxygen species (ROS) overproduction, and increased alpha-synuclein (α-syn) protein production. See Hong, et al., Insulin Resistance Promotes Parkinson's Disease through Aberrant Expression of α-Synuclein, Mitochondrial Dysfunction, and Deregulation of the Polo-Like Kinase 2 Signaling, Cells, v. 9, pp. 740-769 (2020) and Athauda, et al., Insulin Resistance and Parkinson's Disease: A New Target for Disease Modification?, Progress in Neurobiology, v. 145, pp. 98-120 (2016).

Recent studies further reflect that abnormal insulin secretion and impaired insulin signaling resulting therefrom can, and in many instances will, contribute to Parkinson's disease development. See Dierssen, et al., Brain Insulin Resistance in Neurodevelopmental and Neurodegenerative Disorders: Mind the Gap!, Frontiers in Neuroscience, v. 15, p. 730378 (2021).

As also indicated above, recent studies also reflect that there is a strong correlation between abnormal insulin secretion (and insulin resistance-associated) disorders; particularly, type 2 diabetes mellitus, and neurodegenerative disorders; particularly, Parkinson's disease. See, e.g., De Iuliis, et al., Diabetes Mellitus and Parkinson's Disease: Dangerous Liaisons Between Insulin and Dopamine, Neural Regeneration Research, vol. 17.3, pp. 523-533 (2022).

As is well established, insulin comprises seminal neuroprotective properties that protect neurons and glial cells from oxidative damage induced by extracellular reactive oxygen species (ROS), e.g., hydrogen peroxide (H₂O₂). The studies reflect that the neuroprotective properties of insulin are provided by virtue of suppression of the glycogen-synthase kinase 3 (GSK-3) cell signaling pathway, which, when dysregulated, induces seminal neurodegenerative pathophysiological effects, including (i) the formation of Lewy bodies, (ii) ROS-mediated damage and death of dopaminergic neurons, and (iii) induced neuroinflammatory processes.

It is also well established that there is a strong correlation between obesity and type 2 diabetes mellitus (and, hence, neurodegenerative disorders). Indeed, recent research confirms that obesity, i.e., excess visceral fat mass, increases adiposity and results in hypertriglyceridemia, whereby, adipocytes release chemotactic factors, such as monocyte chemoattractant protein-1 (MCP-1) and tumor necrosis factor-α (TNFα), which modulate inflammatory responses in adipose tissue. MCP-1 initiates the migration of monocytes into visceral adipose tissue (VAT) and promotes their differentiation into mature macrophages.

The mature macrophages then secrete large amounts of pro-inflammatory cytokines, e.g., TNFα and IL-1β, and, thereby, (i) increase lipolysis, (ii) decrease insulin-stimulated glucose transporter type-4 (GLUT4) glucose transport in muscle tissue, and (iii) impair triglyceride biosynthesis and adipocyte storage in VAT, which results in an increase in circulating serum triglyceride and oxidized LDL (oxLDL) levels and, thereby, ectopic lipid deposition of toxic fatty acid species (e.g., diacylglycerol and ceramide) in extra-adipose tissue, such as the pancreas. The ectopic lipid deposition of the fatty acid species results in insufficient insulin production and secretion by the pancreas via pancreatic β-cell impairment and insulin resistance by inhibiting insulin-stimulated glucose transport in muscle tissue through activation of protein kinases protein kinase C (PKC), IKKβ, and JNK, which can, and in many instances will, result in type 2 diabetes mellitus. See Guilherme, et al., Adipocyte Dysfunctions Linking Obesity to Insulin Resistance and Type 2 Diabetes, Nature Reviews: Molecular cell Biology, v. 9.5, pp. 367-377 (2008).

As indicated above, in a preferred embodiment, the GLP-1/PYY, GIP and GLP-1/GIP compositions of the invention can, and will, effectively and safely induce secretion of GLP-1 and, thereby, insulin secretion in vivo when delivered to a patient.

According to the invention, the GLP-1/PYY, GIP and GLP-1/GIP compositions of the invention are also adapted to reduce the hepatic secretion of glucose and, thereby, abate hyperglycemia and, thereby, increase systemic insulin sensitivity when delivered to a patient.

As discussed in detail in Applicant's priority U.S. application Ser. No. 18/430,796, which is incorporated by reference herein in its entirety, the GLP-1 secreted by the GLP-1/PYY, GIP and GLP-1/GIP compositions of the invention also decreases the rate of gastric emptying and acid secretion, resulting in reduced appetite and, thereby, induced weight loss. The weight loss also ameliorates hypertriglyceridemia, hyperglycemia and increases systemic insulin sensitivity.

As also indicated above and discussed in detail in priority U.S. application Ser. No. 18/430,796, the GLP-1/PYY and GLP-1/GIP compositions of the invention, when delivered to a patient, also effectively and safely induce secretion of PYY in vivo, which promotes satiety and also decreases the rate of gastric emptying, whereby weight loss and, thereby, increased systemic insulin sensitivity are further induced and hypertriglyceridemia and hyperglycemia are further ameliorated.

The GLP-1/PYY, GIP and GLP-1/GIP compositions of the invention, when delivered to a patient presenting with Parkinson's disease, are thus adapted to effectively and safely treat the Parkinson's disease, and ameliorate at least one seminal physiological risk factor associated with the Parkinson's disease and/or at least one pathophysiological effect associated with the Parkinson's disease.

The GLP-1/PYY, GIP and GLP-1/GIP compositions of the invention, when delivered to a patient presenting with type 2 diabetes mellitus, are thus also adapted to effectively and safely treat the type 2 diabetes mellitus, and ameliorate at least one seminal physiological risk factor associated with the type 2 diabetes mellitus and/or at least one pathophysiological effect associated with the type 2 diabetes mellitus.

The GLP-1/PYY, GIP and GLP-1/GIP compositions of the invention, when delivered to a patient presenting with obesity, are thus also adapted to effectively and safely treat the obesity, and ameliorate at least one seminal physiological risk factor associated with the obesity and/or at least one pathophysiological effect associated with the obesity.

The GLP-1/PYY, GIP and GLP-1/GIP compositions of the invention, when delivered to a patient presenting with Parkinson's disease and type 2 diabetes mellitus, are also adapted to effectively and safely treat the Parkinson's disease and the type 2 diabetes mellitus, and ameliorate at least one seminal physiological risk factor associated with the Parkinson's disease and/or at least one pathophysiological effect associated with the Parkinson's disease, and ameliorate at least one seminal physiological risk factor associated with the type 2 diabetes mellitus and/or at least one pathophysiological effect associated with the type 2 diabetes mellitus.

The GLP-1/PYY, GIP and GLP-1/GIP compositions of the invention, when delivered to a patient presenting with Parkinson's disease, obesity and type 2 diabetes mellitus, are also adapted to effectively and safely treat the Parkinson's disease, obesity and type 2 diabetes mellitus, and ameliorate at least one seminal physiological risk factor associated with the Parkinson's disease and/or at least one pathophysiological effect associated with the Parkinson's disease, ameliorate at least one seminal physiological risk factor associated with the obesity and/or at least one pathophysiological effect associated with the obesity, and ameliorate at least one seminal physiological risk factor associated with the type 2 diabetes mellitus and/or at least one pathophysiological effect associated with the type 2 diabetes mellitus.

According to the invention, the GLP-1/PYY, GIP and GLP-1/GIP compositions of the invention are also adapted to treat Parkinson's disease presented by a patient, and ameliorate at least one seminal physiological risk factor associated with Parkinson's disease and/or at least one pathophysiological effect associated with Parkinson's disease, without the patient also presenting with obesity or type 2 diabetes mellitus.

Treatment of Parkinson's disease, and amelioration of seminal physiological risk factors and pathophysiological effects associated therewith via delivery of the GLP-1/PYY, GIP and GLP-1/GIP compositions of the invention is discussed in detail below.

### Parkinson's Disease

Parkinson disease is a neurodegenerative disorder that adversely affects, among other cells (e.g., glial cells, astrocytes, other neurons, etc.), the dopaminergic neurons in the substantia nigra and, thereby produces motor function-related symptoms, such as tremors, muscle rigidity, and other symptoms that become progressively more debilitating over time.

Indeed, it is established that the progression of Parkinson's disease motor function-related symptoms is attributed to the production of alpha-synuclein (α-syn) proteins in the brain and the resulting accumulation of Lewy bodies formed therefrom, and the associated neuronal dysfunction of dopaminergic neurons.

The resulting reduction in dopamine production due to the dysfunction of the dopaminergic neurons is also a primary driver of neurodegeneration and, hence, progression of Parkinson's disease motor function-related symptoms.

As indicated above and discussed in detail below, recent studies reflect that insulin resistance and, hence, hyperglycemia associated therewith is associated with Parkinson's disease development and progression through (i) mitochondrial dysfunction, (ii) reactive oxygen species (ROS) overproduction, and (iii) increased alpha-synuclein (α-syn) protein production. See Hong, et al., Insulin Resistance Promotes Parkinson's Disease through Aberrant Expression of α-Synuclein, Mitochondrial Dysfunction, and Deregulation of the Polo-Like Kinase 2 Signaling, Cells, v. 9, pp. 740-769 (2020) and Athauda, et al., Insulin Resistance and Parkinson's Disease: A New Target for Disease Modification?, Progress in Neurobiology, v. 145, pp. 98-120 (2016).

The studies also reflect that insulin resistance and, hence, hyperglycemia associated therewith induces a protein glycation reaction resulting in increased production of advanced glycation end products (AGEs), which are produced in the brain from non-enzymatic glycosylation of lipids, lipoproteins and amino acids. The produced AGEs bind to AGE receptors (RAGE) of brain cells and, thereby, induce the production of neuroinflammatory cytokines and, hence, a plurality of neuroinflammatory processes (e.g., maladaptive inflammatory gene expression, NK-KB cell signaling pathway activation, and pro-neuroinflammatory ROS production), which are shown to be associated with an increased production of α-syn production and deposition in the brain via dysregulation of the PI3K/Akt/GSK3β cell signaling pathway. The increased production of α-syn and, hence, accumulation of Lewy bodies results in dopaminergic neuron dysfunction and an attendant reduction in dopamine production in the brain. See De Iuliis, et al., Diabetes Mellitus and Parkinson's Disease: Dangerous Liaisons Between Insulin and Dopamine, Neural Regeneration Research, vol. 17.3, pp. 523-533 (2022).

In most instances, the binding of AGEs to the RAGE receptor of damaged brain cells also induces apoptosis of the damaged cells and, hence, a plurality of pro-neuroinflammatory responses to the apoptosis of the cells and the pro-apoptotic (and pro-neuroinflammatory) cytokines released thereby that promote further dopaminergic neuron dysfunction and reduced dopamine production.

The studies also reflect that insulin resistance and, hence, hyperglycemia associated therewith saturates the polyol pathway and, thereby, abates the production of the reduced form of glutathione (GSH), which is an endogenous compound that neutralizes ROS species and abates neuronal oxidative stress and ROS-induced neuronal damage and attendant mitochondrial dysfunction of dopaminergic neurons. See De Iuliis, et al., Diabetes Mellitus and Parkinson's Disease: Dangerous Liaisons Between Insulin and Dopamine, supra.

As discussed in detail below, in a preferred embodiment, when the GLP-1/PYY, GIP and GLP-1/GIP compositions of the invention are delivered to a patient presenting with Parkinson's disease, the GLP-1/PYY, GIP and GLP-1/GIP compositions effectively and safely treat the Parkinson's disease, i.e., inhibit the formation and progression of the Parkinson's disease, and ameliorate at least one physiological risk factor associated with Parkinson's disease and/or at least one seminal pathophysiological effect associated with Parkinson's disease by inducing the secretion of endogenous GLP-1, which binds to the GLP-1 receptors of dopaminergic neurons.

As indicated above and discussed in detail below, recent studies have shown that when dopaminergic neuron GLP-1 receptors are activated by the secreted endogenous GLP-1 a myriad of seminal neuroprotective activities are induced, including (i) anti-neuroinflammatory processes, (ii) neutralization of overproduced extracellular ROS species, (iii) decreased mitochondrial dysfunction-associated apoptosis of dopaminergic neurons and (iv) decreased production of α-syn and deposition in the brain and, hence, Lewy body formation. See Laurindo, et al., GLP-1 α: Going Beyond Traditional Use., Int. J. Mol. Sci., vol. 23(2), pg. 739 (2022).

Activation of dopaminergic neuron GLP-1 receptors also restores expression of tyrosine hydroxylase, which is a seminal enzyme employed by dopaminergic neurons to synthesize dopamine by catalyzing the conversion of L-tyrosine to L-3,4-dihydroxy-phenylalanine, i.e., L-DOPA, and, thereby, promotes dopamine production. See Mulvaney, et al., GLP-1 Receptor Agonists for Parkinson's Disease, Cochrane Database Syst Rev., vol. 7(7) (2020).

In some embodiments of the invention, the GLP-1/PYY, GIP and GLP-1/GIP compositions of the invention also delay the progression of Parkinson's disease, and ameliorate at least one physiological risk factor associated with Parkinson's disease and/or at least one seminal pathophysiological effect associated with Parkinson's disease, by increasing insulin sensitivity and, hence, ameliorating hyperglycemia associated therewith and, thereby, abating the saturation of the polyol pathway, which increases the production of the reduced form of GSH.

The increased production of reduced GSH results in the increased neutralization of overproduced extracellular ROS species and pro-neuroinflammatory ROS and, thereby, abates neuronal oxidative stress and ROS-induced neuronal damage and attendant mitochondrial dysfunction of dopaminergic neurons.

As indicated above, according to the invention, when the GLP-1/PYY, GIP and GLP-1/GIP compositions of the invention are delivered to a patient presenting with Parkinson's disease, the GLP-1/PYY, GIP and GLP-1/GIP compositions effectively ameliorate at least one physiological risk factor associated with Parkinson's disease, including, without limitation, impaired insulin production, systemic insulin resistance, and increased α-syn protein production and deposition and, hence, Lewy body accumulation.

As also indicated above, when the GLP-1/PYY, GIP and GLP-1/GIP compositions of the invention are delivered to a patient presenting with Parkinson's disease, the GLP-1/PYY, GIP and GLP-1/GIP compositions of the invention also preferably ameliorate at least one pathophysiological effect associated with Parkinson's disease, including, without limitation, neuronal dysfunction, e.g., mitochondrial dysfunction and apoptosis of dopaminergic neurons and glial cells, neuroinflammatory processes and neuronal oxidative stress and ROS-induced neuronal damage and attendant mitochondrial dysfunction of dopaminergic neurons.

According to the invention, the GLP-1/PYY, GIP and GLP-1/GIP compositions of the invention thus can, and will, effectively and safely treat Parkinson's disease presented by a patient, e.g., restoring GLP-1 and insulin mediated neuroprotection, and ameliorate and/or stabilize at least one physiological risk factor and ameliorate and/or stabilize at least one pathophysiological effect associated with Parkinson's disease, by inducing multiple seminal physiological processes, including, without limitation, (i) increasing systemic GLP-1 production, (ii) increasing systemic insulin production, (iii) suppressing the GSK-3 cell signaling pathway, (iv) decreasing α-syn protein production and deposition, (v) increasing reduced GSH production and, hence, ROS neutralization thereby, and (vi) abating one or more seminal neuroinflammatory processes, e.g., maladaptive inflammatory gene expression, NK-κB cell signaling pathway activation, and pro-neuroinflammatory ROS production.

As indicated above, there is also a strong correlation between the physiological risk factors associated with insulin-resistance-induced physiological disorders; particularly, type 2 diabetes mellitus, and Parkinson's disease.

As also indicated above and discussed in detail in Applicant's priority U.S. application Ser. No. 18/430,796, the GLP-1/PYY, GIP and GLP-1/GIP compositions of the invention can, and will, effectively treat type 2 diabetes mellitus via the induced secretion of GLP-1, which reduces appetite and decreases the rate of gastric emptying and acid secretion, which results in reduced appetite and, thereby, induced weight loss. The weight loss also ameliorates hypertriglyceridemia, hyperglycemia and increases systemic insulin sensitivity.

As also indicated above and discussed in detail in priority U.S. application Ser. No. 18/430,796, the induced secretion of PYY in vivo by the GLP-1/PYY, GIP and GLP-1/GIP compositions of the invention promotes satiety and also decreases the rate of gastric emptying, whereby weight loss and, thereby, increased systemic insulin sensitivity are further induced and hypertriglyceridemia and hyperglycemia are further ameliorated.

Thus, in some embodiments of the invention, the GLP-1/PYY, GIP and GLP-1/GIP compositions of the invention, when delivered to a patient presenting with Parkinson's disease and type 2 diabetes mellitus will effectively and safely (i) treat the Parkinson's disease, (ii) ameliorate and/or stabilize at least one physiological risk factor of Parkinson's disease, and (iii) preferably ameliorate and/or stabilize at least one pathophysiological effect associated with Parkinson's disease, and (iv) treat the type 2 diabetes mellitus, (v) ameliorate and/or stabilize at least one physiological risk factor associated with type 2 diabetes mellitus, and (vi) preferably ameliorate and/or stabilize at least one pathophysiological effect associated with type 2 diabetes mellitus.

As also indicated above, there is also a strong correlation between obesity and type 2 diabetes mellitus. See, e.g., Guilherme, et al., Adipocyte Dysfunctions Linking Obesity to Insulin Resistance and Type 2 Diabetes, Nature Reviews: Molecular Cell Biology, v. 9.5, pp. 367-377 (2008).

Indeed, as additionally indicated above, recent research confirms that obesity, i.e., excess visceral fat mass, increases adiposity and results in hypertriglyceridemia, whereby, adipocytes release chemotactic factors, such as monocyte chemoattractant protein-1 (MCP-1) and tumor necrosis factor-α (TNFα), which modulate inflammatory responses in adipose tissue. MCP-1 initiates the migration of monocytes into visceral adipose tissue (VAT) and promotes their differentiation into mature macrophages.

The mature macrophages then secrete large amounts of pro-inflammatory cytokines, e.g., TNFα and IL-1β, and, thereby, (i) increase lipolysis, (ii) decrease insulin-stimulated glucose transporter type-4 (GLUT4) glucose transport in muscle tissue, and (iii) impair triglyceride biosynthesis and adipocyte storage in VAT, which results in an increase in circulating serum triglyceride and oxidized LDL (oxLDL) levels and, thereby, ectopic lipid deposition of toxic fatty acid species (e.g., diacylglycerol and ceramide) in extra-adipose tissue, such as the pancreas. The ectopic lipid deposition of the fatty acid species results in (i) insufficient insulin production and secretion by the pancreas via pancreatic β-cell impairment and (ii) insulin resistance by inhibiting insulin-stimulated glucose transport in muscle tissue through activation of protein kinases protein kinase C (PKC), IKKβ, and JNK, which can, and in many instances will, result in type 2 diabetes mellitus.

As also indicated above and discussed in detail in Applicant's priority U.S. application Ser. Nos. 18/430,796 and 18/980,129, the GLP-1/PYY, GIP and GLP-1/GIP compositions of the invention, when delivered to a patient presenting with obesity can, and also will, effectively treat obesity via induced secretion of GLP-1 and PYY in vivo.

Thus, according to the invention, the GLP-1/PYY, GIP and GLP-1/GIP compositions of the invention can, and will, effectively and safely treat Parkinson's disease, i.e., inhibit the formation and progression of the Parkinson's disease, and ameliorate at least one physiological risk factor associated with the Parkinson's disease and/or at least one seminal pathophysiological effect associated with the Parkinson's disease when delivered to a patient presenting with Parkinson's disease, and, if the patient also presents with obesity or type 2 diabetes mellitus, also treat the obesity and ameliorate at least one physiological risk factor associated with the obesity and treat the type 2 diabetes mellitus, and ameliorate at least one physiological risk factor associated with the type 2 diabetes mellitus and/or at least one seminal pathophysiological effect associated with the type 2 diabetes mellitus.

As indicated above, in a preferred embodiment, the noted GLP-1/PYY, GIP and GLP-1/GIP compositions preferably comprise at least one of the natural insulin modulating compounds or ligands referenced above that is adapted to bind to and activate at least one insulin modulating receptor; specifically, olfactory receptor OR51E1, olfactory receptor OR1A1 or olfactory receptor OR2C1, and at least one free fatty acid receptor; specifically, free fatty acid receptor FFAR1 or free fatty acid receptor FFAR4, and, in some instances, at least one transient receptor; specifically, transient receptor TRPA1.

In some embodiments, the noted GLP-1/PYY, GIP and GLP-1/GIP compositions preferably comprise a plurality of the natural insulin modulating compounds or ligands referenced above that are adapted to bind to and activate a plurality of the insulin modulating receptors, preferably, olfactory receptor OR51E1, olfactory receptor OR1A1 or olfactory receptor OR2C1, and at least one free fatty acid receptor; specifically, free fatty acid receptor FFAR1 or free fatty acid receptor FFAR4, and, in some instances, at least one transient receptor; specifically, transient receptor TRPA1.

In some embodiments, the noted GLP-1/PYY, GIP and GLP-1/GIP compositions preferably comprise a plurality of the natural insulin modulating compounds or ligands referenced above that are specifically adapted to bind to and activate at least olfactory receptor OR51E1 and free fatty acid receptor FFAR1 or free fatty acid receptor FFAR4.

As indicated above, in some embodiment of the invention, the noted GLP-1/PYY, GIP and GLP-1/GIP compositions comprise at least one of the natural dopamine modulating compounds and ligands referenced above that is adapted to bind to and activate at least one of the dopamine modulating receptors, i.e., olfactory receptor OR2A4, olfactory receptor OR51E1, olfactory receptor OR51E2, transient receptor TRPC4, taste receptor TASIR3 and trace amine receptor TAAR5.

According to the invention, the GLP-1/PYY, GIP and GLP-1/GIP compositions of the invention can also comprise at least one natural dopamine modulating compound or ligand that is adapted to bind to and activate olfactory receptor family 2 subfamily A member 4 (OR2A4) of endogenous cells, such as, without limitation, cyclohexyl salicylate (CHS) and sandacanol, wherein, when the GLP-1/PYY, GIP and GLP-1/GIP compositions are delivered to a patient presenting with Parkinson's disease, the GLP-1/PYY, GIP and GLP-1/GIP compositions induce dopaminergic activation of olfactory receptor OR2A4 in vivo, whereby dopamine synthesis is induced and, thereby, dopamine secretion is also modulated and, hence, treatment of the Parkinson's disease is further facilitated and/or at least one seminal pathophysiological effect associated with Parkinson's disease is further ameliorated.

According to the invention, the GLP-1/PYY, GIP and GLP-1/GIP compositions of the invention can also comprise at least one natural dopamine modulating compound or ligand that is adapted to bind to and activate taste 1 receptor member 3 (TAS1R3) of endogenous cells, such as, without limitation, brazzein, monellin, xylitol and sorbitol, wherein, when the GLP-1/PYY, GIP and GLP-1/GIP compositions are delivered to a patient presenting with Parkinson's disease, the GLP-1/PYY, GIP and GLP-1/GIP compositions induce activation of taste receptor TASIR3 in vivo, whereby dopamine synthesis is similarly induced and, thereby, dopamine secretion is also modulated and, hence, treatment of the Parkinson's disease is further facilitated and/or at least one seminal pathophysiological effect associated with Parkinson's disease is further ameliorated.

According to the invention, the GLP-1/PYY, GIP and GLP-1/GIP compositions of the invention can also comprise at least one natural dopamine modulating compound or ligand that is adapted to bind to and activate trace amine associated receptor 5 (TAAR5) of endogenous cells, such as, without limitation, trimethylamine (TMA) and N,N-dimethylethylamine, wherein, when the GLP-1/PYY, GIP and GLP-1/GIP compositions are delivered to a patient presenting with Parkinson's disease, the GLP-1/PYY, GIP and GLP-1/GIP compositions induce dopaminergic activation of trace amine receptor TAAR5 in vivo, whereby dopamine synthesis and secretion by dopaminergic neurons is similarly induced and, hence, treatment of the Parkinson's disease is further facilitated and/or at least one seminal pathophysiological effect associated with Parkinson's disease is further ameliorated.

Various exemplar and, hence, non-limiting, embodiments of GLP-1/PYY and GLP-1/GIP compositions of the invention that are adapted to directly treat Parkinson's disease, and ameliorate at least one physiological risk factor and/or at least one seminal pathophysiological effect induced by the Parkinson's disease, and concurrently (i) treat Parkinson's disease, and ameliorate at least one physiological risk factor and/or at least one seminal pathophysiological effect induced by the Parkinson's disease and (ii) treat type 2diabetes mellitus, and ameliorate at least one physiological risk factor and/or at least one seminal pathophysiological effect induced by the type 2 diabetes mellitus, are set forth below.

In one embodiment of the invention, a GLP-1/PYY secretion composition for treating a neurodegenerative disorder presented by a patient comprises at least one receptor activating compound adapted to bind to and activate at least one receptor selected from the group comprising olfactory receptor OR51E1, olfactory receptor OR1A1, olfactory receptor OR2C1 and olfactory receptor OR10J5,
the GLP-1/PYY secretion composition adapted to induce GLP-1 and PYY secretion, and, thereby, increased insulin secretion in vivo, whereby the neurodegenerative disorder is treated when the GLP-1/PYY secretion composition is delivered to the patient.

In a preferred embodiment, the GLP-1/PYY secretion composition is further adapted to ameliorate at least one at least one risk factor associated with the neurodegenerative disorder when the GLP-1/PYY secretion composition is delivered to the patient.

In some embodiments, the GLP-1/PYY secretion composition is further adapted to ameliorate at least one seminal pathophysiological effect associated with the neurodegenerative disorder when the GLP-1/PYY secretion composition is delivered to the patient.

In a preferred embodiment, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating a neurodegenerative disorder presented by a patient comprises at least one receptor activating compound adapted to bind to and activate at least one receptor selected from the group comprising olfactory receptor OR51E1 and olfactory receptor OR1A1,
the GLP-1/PYY secretion composition adapted to induce GLP-1 and PYY secretion, and, thereby, increased insulin secretion in vivo, whereby the neurodegenerative disorder is treated when the GLP-1/PYY secretion composition is delivered to the patient.

In a preferred embodiment, the GLP-1/PYY secretion composition is further adapted to ameliorate at least one at least one risk factor associated with the neurodegenerative disorder when the GLP-1/PYY secretion composition is delivered to the patient.

In some embodiments, the GLP-1/PYY secretion composition is further adapted to ameliorate at least one seminal pathophysiological effect associated with the neurodegenerative disorder when the GLP-1/PYY secretion composition is delivered to the patient.

In some embodiments, the receptor activating compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the receptor activating compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 30.0 μM.

In a preferred embodiment, the receptor activating compound is adapted to induce at least 50% activation of at least olfactory receptor OR51E1 in vivo when the GLP-1/PYY secretion composition is delivered to the patient.

In another embodiment of the invention, a GLP-1/PYY secretion composition for treating a neurodegenerative disorder presented by a patient comprises a plurality of receptor activating compounds adapted to bind to and activate at least olfactory receptor OR51E1 and olfactory receptor OR1A1,
the GLP-1/PYY secretion composition adapted to induce GLP-1 and PYY secretion, and, thereby, increased insulin secretion in vivo, whereby the neurodegenerative disorder is treated when the GLP-1/PYY secretion composition is delivered to the patient.

In a preferred embodiment, the GLP-1/PYY secretion composition is further adapted to ameliorate at least one at least one risk factor associated with the neurodegenerative disorder when the GLP-1/PYY secretion composition is delivered to the patient.

In some embodiments, the GLP-1/PYY secretion composition is further adapted to ameliorate at least one seminal pathophysiological effect associated with the neurodegenerative disorder when the GLP-1/PYY secretion composition is delivered to the patient.

In a preferred embodiment, the plurality of receptor activating compounds comprise eugenol, 3-methylpentanoic acid and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 30.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 15.0 μM.

In a preferred embodiment, at least one of the plurality of receptor activating compounds is adapted to induce at least 50% activation of at least olfactory receptor OR51E1 in vivo when the GLP-1/PYY secretion composition is delivered to the patient.

In another embodiment of the invention, a GLP-1/PYY secretion composition for treating a neurodegenerative disorder and an insulin-resistance-induced physiological disorder presented by a patient comprises a plurality of receptor activating compounds adapted to bind to and activate at least olfactory receptor OR51E1 and olfactory receptor OR1A1,
the GLP-1/PYY secretion composition similarly adapted to induce GLP-1 and PYY secretion, and, thereby, increased insulin secretion in vivo, whereby the neurodegenerative disorder and insulin-resistance-induced physiological disorder are treated when the GLP-1/PYY secretion composition is delivered to the patient.

In a preferred embodiment, the GLP-1/PYY secretion composition is further adapted to ameliorate at least one risk factor associated with the neurodegenerative disorder when the GLP-1/PYY secretion composition is delivered to the patient.

In a preferred embodiment, the neurodegenerative disorder comprises Parkinson's disease.

In some embodiments, the insulin-resistance-induced physiological disorder comprises type 2 diabetes mellitus.

In a preferred embodiment, the plurality of receptor activating compounds comprise eugenol and geraniol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the geraniol comprises an EC₅₀ value of at least approximately 0.1 μM in the GLP-1/PYY secretion composition.

In some embodiments, the geraniol comprises an EC₅₀ value in the GLP-1/GIP secretion composition in the range of approximately 1.0 μM to approximately 15.0 μM.

In a preferred embodiment, at least one of the receptor activating compounds is adapted to induce at least 50% activation of at least OR51E1 in vivo when the GLP-1/PYY secretion composition is delivered to the patient.

In another embodiment of the invention, a GLP-1/GIP secretion composition for treating a neurodegenerative disorder presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby the neurodegenerative disorder is treated when the GLP-1/GIP secretion composition is delivered to the patient.

In a preferred embodiment, the GLP-1/GIP secretion composition is further adapted to ameliorate at least one risk factor associated with the neurodegenerative disorder when the GLP-1/GIP secretion composition is delivered to the patient.

In some embodiments, the GLP-1/GIP secretion composition is further adapted to ameliorate at least one seminal pathophysiological effect associated with the neurodegenerative disorder when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM. In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In another embodiment of the invention, a GLP-1/GIP secretion composition for treating a neurodegenerative disorder comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5,
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1, and
(iii) at least a third receptor activating compound adapted to bind to and activate olfactory receptor OR51E2,
the GLP-1/GIP secretion composition adapted to induce GLP-1 and GIP secretion, and, thereby, increased insulin secretion in vivo, whereby the neurodegenerative disorder is treated when the GLP-1/GIP secretion composition is delivered to a patient.

In a preferred embodiment, the GLP-1/GIP secretion composition is further adapted to ameliorate at least one risk factor associated with the neurodegenerative disorder when the GLP-1/GIP secretion composition is delivered to the patient.

In some embodiments, the GLP-1/GIP secretion composition is further adapted to ameliorate at least one seminal pathophysiological effect associated with the neurodegenerative disorder when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In some embodiments, the third receptor activating compound comprises a fourth compound selected from the group comprising β-ionone, propionic acid and acetate.

In some embodiments, the fourth compound comprises β-ionone.

In some embodiments, the β-ionone comprises an EC₅₀ value of at least approximately 0.1 μM in the GLP-1/GIP secretion composition.

In some embodiments, the fourth compound comprises propionic acid.

In some embodiments, the propionic acid comprises an EC₅₀ value of at least approximately 0.1 μM in the GLP-1/GIP secretion composition.

In a preferred embodiment, at least one of the first receptor activating compounds is adapted to induce at least 50% activation of at least olfactory receptor olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, at least one of the third receptor activating compounds is adapted to induce at least 50% activation of olfactory receptor OR51E2 in vivo when the GLP-1/GIP secretion composition is delivered to the patient.

In another embodiment of the invention, a GLP-1/GIP secretion composition for treating a neurodegenerative disorder comprises:
(i) at least a first receptor activating compound adapted to bind to and activate at least a first receptor selected from the group comprising olfactory receptor family 51 subfamily E member 1 (OR51E1), olfactory receptor family 1 subfamily A member 1 (OR1A1), olfactory receptor family 2 subfamily C member 1 (OR2C1), and olfactory receptor family 10 subfamily J member 5 (OR10J5),
(ii) at least a second receptor activating compound adapted to bind to and activate at least a second receptor selected from the group comprising free fatty acid receptor 1 (FFAR1), free fatty acid receptor 4 (FFAR4), olfactory receptor family 2 subfamily W member 1 (OR2W1), olfactory receptor family 2 subfamily B member 11 (OR2B11), olfactory receptor family 2 subfamily J member 3 (OR2J3), and transient receptor potential cation channel subfamily A member 1 (TRPA1, and
(iii) at least a third receptor activating compound adapted to bind to and activate short transient receptor potential channel 4 (TRPC4),
the GLP-1/GIP secretion composition adapted to modulate insulin and dopamine secretion in vivo, whereby the neurodegenerative disorder is treated when the GLP-1/GIP secretion composition is delivered to a patient.

In a preferred embodiment, the GLP-1/GIP secretion composition is further adapted to ameliorate at least one risk factor associated with the neurodegenerative disorder when the GLP-1/GIP secretion composition is delivered to the patient.

In some embodiments, the GLP-1/GIP secretion composition is further adapted to ameliorate at least one seminal pathophysiological effect associated with the neurodegenerative disorder when the GLP-1/GIP secretion composition is delivered to the patient.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In some embodiments, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In some embodiments, the third receptor activating compound comprises a fourth compound selected from the group comprising (−)-englerin A and choline.

In some embodiments, the fourth compound comprises (−)-englerin A.

In some embodiments, the (−)-englerin A comprises an EC₅₀ value of at least approximately 0.1 μM in the GLP-1/GIP secretion composition.

In some embodiments, the (−)-englerin A comprises an EC₅₀ value in the GLP-1/GIP secretion composition in the range of approximately 0.003 μM to approximately 0.1 μM.

In a preferred embodiment, at least one of the first receptor activating compounds is adapted to induce at least 50% activation of at least olfactory receptor olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In a preferred embodiment, at least one of the third receptor activating compounds is adapted to induce at least 50% activation of transient receptor TRPC4 in vivo when the GLP-1/GIP secretion composition is delivered to the patient.

In another embodiment of the invention, a GLP-1/GIP secretion composition for treating a neurodegenerative disorder and an insulin-resistance-induced physiological disorder presented by a patient comprises:
(i) at least a first receptor activating compound adapted to bind to and activate at least one first receptor selected from the group comprising olfactory receptor OR51E1, olfactory receptor OR1A1, olfactory receptor OR2C1 and olfactory receptor OR10J5, and
(ii) at least a second receptor activating compound adapted to bind to and activate at least one second receptor selected from the group comprising free fatty acid receptor FFAR1, free fatty acid receptor FFAR4, olfactory receptor OR2W1, olfactory receptor OR2B11, olfactory receptor OR2J3 and transient receptor TRPA1,
the GLP-1/GIP secretion composition adapted to induce GLP-1 and GIP secretion, and, thereby, increased insulin secretion in vivo, whereby the neurodegenerative disorder and insulin-resistance-induced physiological disorder are treated when the GLP-1/GIP secretion composition is delivered to a patient.

In a preferred embodiment, the GLP-1/GIP secretion composition is further adapted to ameliorate at least one risk factor associated with the neurodegenerative disorder when the GLP-1/GIP secretion composition is delivered to the patient.

In a preferred embodiment, the plurality of physiological disorders comprises a neurodegenerative disorder and an insulin-resistance-induced physiological disorder.

In a preferred embodiment, the neurodegenerative disorder comprises Parkinson's disease.

In some embodiments, the insulin-resistance-induced physiological disorder comprises type 2 diabetes mellitus.

In some embodiments, the first receptor activating compound comprises a first compound selected from the group comprising eugenol and 3-methylpentanoic acid.

In some embodiments, the first compound comprises eugenol.

In some embodiments, the eugenol comprises an EC₅₀ value of at least approximately 0.1 μM in the GLP-1/PYY secretion composition.

In some embodiments, the eugenol comprises an EC₅₀ value in the GLP-1/GIP secretion composition in the range of approximately 1.0 μM to approximately 15.0 μM.

In some embodiments, the first compound comprises 3-methylpentanoic acid.

In some embodiments, the 3-methylpentanoic acid comprises an EC₅₀ value of at least approximately 0.1 μM in the GLP-1/PYY secretion composition.

In some embodiments, the 3-methylpentanoic acid comprises an EC₅₀ value in the GLP-1/GIP secretion composition in the range of approximately 10.0 μM to approximately 50.0 μM.

In a preferred embodiment, the second receptor activating compound comprises a second compound selected from the group comprising a medium-chain free fatty acid, a long-chain free fatty acid and an omega-3 polyunsaturated fatty acid.

In some embodiments, the second compound comprises a medium-chain free fatty acid.

In some embodiments, the medium-chain free fatty acid comprises lauric acid.

In some embodiments, the lauric acid comprises an EC₅₀ value of at least approximately 0.05 μM in the GLP-1/GIP secretion composition.

In some embodiments, the lauric acid comprises an EC₅₀ value in the GLP-1/GIP secretion composition in the range of approximately 0.05 μM to approximately 50.0 μM.

In some embodiments, the second receptor activating compound comprises a third compound selected from the group comprising cinnamaldehyde and cis-3-hexen-1-ol.

In some embodiments, the third compound comprises cinnamaldehyde.

In some embodiments, the cinnamaldehyde comprises an EC₅₀ value of at least approximately 0.1 μM in the GLP-1/GIP secretion composition.

In some embodiments, the cinnamaldehyde comprises an EC₅₀ value in the GLP-1/GIP secretion composition in the range of approximately 0.1 μM to approximately 2500.0 μM.

In a preferred embodiment, at least one of the first receptor activating compounds is adapted to induce at least 50% activation of at least olfactory receptor OR51E1 in vivo when the GLP-1/GIP secretion composition is delivered to the patient.

In a preferred embodiment, at least one of the second receptor activating compounds is adapted to induce at least 50% activation of free fatty acid receptor FFAR1 or FFAR4 in vivo when the GLP-1/GIP secretion composition is delivered to the patient.

In some embodiments of the invention, there are thus also provided methods for treating a neurodegenerative disorder presented by a patient.

In some embodiments, a method for treating a neurodegenerative disorder presented by a patient comprises the steps of:
(a) providing one of the GLP-1/PYY secretion compositions of the invention; and
(b) delivering the GLP-1/PYY secretion composition to the patient, wherein insulin secretion of the patient is modulated in vivo, whereby the neurodegenerative disorder is effectively treated.

In some embodiments, a method for treating a neurodegenerative disorder presented by a patient comprises the steps of:
(a) providing one of the GLP-1/GIP secretion compositions of the invention; and
(b) delivering the GLP-1/GIP secretion composition to the patient, wherein insulin secretion of the patient is modulated in vivo, whereby the neurodegenerative disorder is effectively treated.

In some embodiments, a method for treating a neurodegenerative disorder presented by a patient comprises the steps of:
(a) providing one of the GLP-1/GIP secretion compositions of the invention; and
(b) delivering the composition to the patient, wherein insulin and dopamine secretion of the patient are modulated in vivo, whereby the neurodegenerative disorder is treated.

In some embodiments of the invention, there are also provided methods for treating a neurodegenerative disorder and an insulin-resistance-induced physiological disorder presented by a patient.

In some embodiments, a neurodegenerative disorder and an insulin-resistance-induced physiological disorder presented by a patient comprises the steps of:
(a) providing one of the GLP-1/GIP secretion compositions of the invention; and
(b) delivering the composition to the patient, wherein insulin and dopamine secretion of the patient are modulated in vivo, whereby the neurodegenerative disorder and insulin-resistance-induced physiological disorder are treated.

As will readily be appreciated by one having ordinary skill in the art, the present invention provides numerous advantages compared to prior art formulations and methods for enhancing cell function and, thereby, mental function and acuity. Among the advantages are the following:
The provision of natural compounds and ligands, and compositions comprising same, for treating physiological disorders; particularly, insulin-resistance-induced physiological disorders, such as type 2 diabetes mellitus, and neurodegenerative disorders, such as Parkinson's disease, which substantially reduce or overcome the drawbacks and disadvantages associated with administration of GLP-1 analogs and dual GLP-1/GIP analogs to a patient presenting with a physiological disorder;
The provision of natural compounds and ligands, and compositions comprising same, which effectuate OR-mediated, free fatty acid receptor-mediated, and transient potential ion channel-mediated secretion of endogenous GLP-1 PYY and GIP in a patient presenting with a physiological disorder, without the undesirable side effects associated with delivery of a GLP-1 analog and/or a dual GLP-1/GIP analog to patients;
The provision of natural compounds and ligands, and compositions comprising same, which, when administered to a patient, effectively and safely modulate the patient's systemic insulin secretion in vivo, and can be administered to the patient via oral, sublingual, inhalation, intranasal, epidural, intracerebral, transdermal, topical, and injection administration means;
The provision of natural compounds and ligands, and compositions comprising same, which, when administered to a patient effectively and safely modulate dopamine synthesis and, thereby, dopamine secretion in vivo, and can be administered to the patient via oral, sublingual, inhalation, intranasal, epidural, intracerebral, transdermal, topical, and injection administration means;
The provision of natural compounds and ligands, and compositions comprising same, which, when administered to a patient presenting with an insulin-resistance-induced physiological disorder; particularly, type 2 diabetes mellitus, effectively and safely modulate the patient's insulin secretion in vivo, whereby the insulin-resistance-induced physiological disorder is effectively treated and at least one risk factor associated with the insulin-resistance-induced physiological disorder is ameliorated with minimal, if any, adverse side effects;
The provision of natural compounds and ligands, and compositions comprising same, which, when administered to a patient presenting with a neurodegenerative disorder; particularly, Parkinson's disease, effectively and safely modulate the patient's insulin secretion in vivo, whereby the neurodegenerative disorder is effectively treated and at least one risk factor associated with the neurodegenerative disorder is ameliorated with minimal, if any, adverse side effects;
The provision of natural compounds and ligands, and compositions comprising same, which, when administered to a patient presenting with a neurodegenerative disorder; particularly, Parkinson's disease, effectively and safely modulate the patient's dopamine secretion in vivo, whereby the neurodegenerative disorder is effectively treated and at least one risk factor associated with the neurodegenerative disorder is ameliorated with minimal, if any, adverse side effects; and
The provision of natural compounds and ligands, and compositions comprising same, which, when administered to a patient presenting with an insulin-resistance-induced physiological disorder; particularly, type 2 diabetes mellitus, and a neurodegenerative disorder; particularly, Parkinson's disease, effectively and safely modulate the patient's insulin and dopamine secretion in vivo, whereby the neurodegenerative disorder and/or neurodegenerative disorder is effectively treated and at least one risk factor associated with the neurodegenerative disorder or neurodegenerative disorder is ameliorated with minimal, if any, adverse side effects;

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

1. A composition for treating a neurodegenerative disorder, comprising:
(i) at least a first receptor activating compound adapted to bind to and activate at least a first receptor selected from the group consisting of olfactory receptor family 51 subfamily E member 1 (OR51E1), olfactory receptor family 1 subfamily A member 1 (OR1A1), olfactory receptor family 2 subfamily C member 1 (OR2C1) and olfactory receptor family 10 subfamily J member 5 (OR10J5),
(ii) at least a second receptor activating compound adapted to bind to and activate at least a second receptor selected from the group consisting of free fatty acid receptor 1 (FFAR1), free fatty acid receptor 4 (FFAR4), olfactory receptor family 2 subfamily W member 1(OR2W1), olfactory receptor family 2 subfamily B member 11 (OR2B11), olfactory receptor family 2 subfamily J member 3 (OR2J3) and transient receptor potential cation channel subfamily A member 1 (TRPA1), and
(iii) at least a third receptor activating compound adapted to activate short transient receptor potential channel 4 (TRPC4),
said composition adapted to modulate insulin and dopamine secretion in vivo, whereby said neurodegenerative disorder is treated when said composition is delivered to a patient presenting with said neurodegenerative disorder.

2. The composition of claim 1, wherein said composition is further adapted to ameliorate at least one risk factor of said neurodegenerative disorder when said composition is said delivered to said patient.

3. The composition of claim 1, wherein said composition is further adapted to ameliorate at least one seminal pathophysiological effect induced by said neurodegenerative disorder when said composition is said delivered to said patient.

4. The composition of claim 1, wherein said neurodegenerative disorder comprises Parkinson's disease.

5. The composition of claim 1, wherein said first receptor activating compound is selected from the group consisting of eugenol, 3-methylpentanoic acid and geraniol.

6. The composition of claim 5, wherein said first receptor activating compound comprises said eugenol.

7. The composition of claim 6, wherein said eugenol comprises a first EC₅₀ concentration in said composition of at least 0.1 μM.

8. The composition of claim 7, wherein said first EC₅₀ concentration of said eugenol in said composition is in the range from about 1.0 μM to about 15.0 μM.

9-11. (canceled)

12. The composition of claim 1, wherein said second receptor activating compound is selected from the group consisting of a medium-chain free fatty acid, a long-chain free fatty acid and an omega-3 polyunsaturated fatty acid.

13. The composition of claim 12, wherein said medium-chain free fatty acid comprises lauric acid.

14. The composition of claim 13, wherein said lauric acid comprises a second EC₅₀ concentration in said composition of at least 0.05 μM.

15. The composition of claim 14, wherein said second EC₅₀ concentration of said lauric acid in said composition is in the range from about 0.05 μM to about 50.0 μM.

16. The composition of claim 1, wherein said third receptor activating compound is selected from the group consisting of cinnamaldehyde and cis-3-hexen-1-ol.

17. The composition of claim 16, wherein said third receptor activating compound comprises said cinnamaldehyde.

18. The composition of claim 17, wherein said cinnamaldehyde comprises a third EC₅₀ concentration in said composition of at least 0.1 μM.

19. The composition of claim 18, wherein said third EC₅₀ concentration of said cinnamaldehyde in said composition is in the range from about 0.1 μM to about 2500.0 μM.

20-23. (canceled)

24. The composition of claim 1, wherein said first receptor activating compound is adapted to induce at least 50% activation of at least said olfactory receptor OR51E1 in vivo when said composition is said delivered to said patient.

25. The composition of claim 1, wherein said third receptor activating compound is adapted to induce at least 50% activation of at least said transient receptor TRPC4 in vivo when said composition is said delivered to said patient.
